(19) United States
(12) Patent Application Publication
(10) Pub. No.: US 2018/0169019 A1
(43) Pub. Date: Jun. 21, 2018

(54) PROCESS FOR THE PREPARATION OF POROUS MICROPARTICLES

(71) Applicants: BAYER PHARMA AKTIENGESELLSCHAFT, Berlin (DE); SHENYANG PHARMACEUTICAL UNIVERSITY, Shenyang, Liaoning (CN)

(72) Inventors: Uwe MUENSTER, Wülfrath (DE); Eva-Maria BECKER-PELSTER, Wuppertal (DE); Martin ROSENBRUCH, Düsseldorf (DE); Shirui MAO, Shenyang, Shenyang, Liaoning (CN); Ni RUI, Lianyunguang, Jiangsu (CN); Zhenglin LIANG, Jiangmen, Guangdong (CN)

(21) Appl. No.: 15/735,421

(22) PCT Filed: Jun. 7, 2016
(86) PCT No.: PCT/EP2016/062884

§ 371 Date: Dec. 11, 2017

(30) Foreign Application Priority Data
Jun. 12, 2015 (CN) PCT/CN2015/000400

(51) Int. Cl. A61K 9/16; A61K 31/197; A61K 31/58; A61K 9/00

(52) U.S. Cl. CPC A61K 9/1694; A61K 9/0075; A61K 31/58; A61K 31/197

(57) ABSTRACT

The invention relates to a single-emulsion based process using polyvinylpyrrolidone as a porogenic agent for the preparation of porous microparticles for inhalation formulations for pulmonary drug delivery as well as the microparticles and the pharmaceutical compositions produced hereof.

The invention relates to the preparation of porous microparticles for inhalation formulations for pulmonary drug delivery as well as the microparticles and the dry powder formulations produced hereof.

Respiratory drug delivery has drawn great attention in recent years since this route can be utilized for both local and systemic treatments. It is extremely suitable for the local treatment of lung diseases such as asthma, fibrosis, cystic fibrosis, pulmonary arterial hypertension, chronic obstructive pulmonary disease (COPD), lung cancers and lung metastases, with the advantages of a targeted local lung action, very thin diffusion path to the blood stream and rich vasculature, rapid onset of therapeutic action, relatively low metabolic activity and fewer systemic side effects than oral therapy. Also a systemic delivery of drugs via the lung and particularly the alveolar regions is an attractive therapeutic concept due to the enormous absorption surface area and also extensive vascularization as well as the aforementioned relatively low metabolic activity.

Various review articles have recently been published covering the field of pulmonary drug delivery and particle engineering technologies [Liang Z., Ni R., Zhou J. Mao S., Drug Discov Today 20 (3), 380-389 (2015); Loira-Pastoriza C., Todoroff J., Vanbever, R., Adv. Drug Deliver. Rev. 75, 81-91 (2014); Rubin K. B., Williams R. W., Adv. Drug Deliver. Rev. 75, 141-148 (2014); Ungaro F., D'Angelo I., Miro A., La Rotonda M. I., Quaglia F., J. Pharm. Pharmacol. 64, 1217-1235 (2012); Chow A. H. L., Tong H. H. Y., Chattopadhyay P., Shekunov B. Y., Pharmaceut. Res. 24 (3) (2007); Patton J. S., Nat. Rev., 6, 67-74 (2007)].

The key features of the formulation, such as the size and geometry of the particles, will have significant impact on the likelihood of being deposited in the targeted regions of the lung. Lung deposition is furthermore influenced by flow and aerolization properties, the mode of inhalation and the inhalation device. Inhalation dosage technology has therefore primarily focused on two parallel development pathways: Fabrication of novel inhaler devices with enhanced efficiency and/or improvement of the existing inhalation formulations via advanced particle engineering strategies. An important determinant of aerosol deposition is the aerodynamic particle size, often expressed as the mass median aerodynamic diameter (MMAD) of a group of particles, where particles with an MMAD in the range of 1-5 μm inhaled at slow flow are more likely to deposit in the more distal parts of the lung, while those in the range of 5-10 μm will deposit proximally in the oral pharynx and tracheobronchial tree, and those larger than 10 μm will likely deposit in the mouth. Other desirable product characteristics constitute a high fine particle fraction (FPF), and emitted dose (ED), high dose consistency and uniformity and, ideally, independence of the type of device and inhalation flow rate. Apart from the correct aerodynamic particle size the particles should have a relatively narrow particle size distribution (PSD) and should be readily aerosolizable at relatively low aerodynamic dispersion forces. Additionally, the requirement of physical and chemical stability implies that storage must not have a significant effect on the drug's physical form (e.g., crystallinity, polymorphism), PSD and/or the dose content uniformity.

Most current dry powder inhalation products are formulated with a drug carrier, commonly lactose, which also serves as a bulking agent and is mostly simply blended with the micronized drugs. Current formulations are however often not ideal in particular aspects, as they may deliver inaccurate doses, require frequent dosing or lose significant amounts of pharmaceutically active agent in the delivery process. Also, the therapeutic efficacy is often limited by a rapid lung absorption, mucociliary clearance, or by macrophage uptake of inhaled particles, which makes it difficult to maintain a therapeutic level at the target site long enough to allow clinically practical dosing. One of the major challenges in pulmonary drug delivery is therefore how to control the pharmacokinetics of inhaled drugs beyond a few hours. In addition, patients often show a non-regular and infrequent inhalation of their prescribed pharmaceuticals. An inhalable formulation with a sustained-release profile, which would allow reducing the administration intervals from two to four times daily down to once daily or even weekly, might therefore also help to enhance patient compliance. Although sustained-drug release in the lungs presents high potential to improve the therapeutic efficacy and safety of inhaled drugs, there is not yet any pulmonary sustained-release formulation available on the market. Only a few pulmonary sustained-release formulations are in clinical development and all are in the form of liposomes [Loira-Pastoriza C., Todoroff J., Vanbever R., Adv. Drug Deliver. Rev. 75, 81-91 (2014)].

Phagocytosis mechanism is size-dependent, with particles 1-5 μm in size being optimum for uptake by macrophages. Unfavorably this is overlapping the optimum range of MMAD for efficient pulmonary drug delivery. It has been found that large porous microparticles with high geometric diameters (10-20 μm) and low bulk densities (˜0.4 g/cm³) show reduced clearance while keeping a favorable MMAD for deep lung deposition [Edwards D. A., Hames J., Caponetti G., Hrkach J., Abdelaziz B.-J., Eskew M. L., Mintzes J., Deaver D., Lotan N., Langer R., Science 276, 1868-1871 (1997)]. In general, porosity of the particles is not only desired to decrease the density of the particles and to control the particle aerodynamics, but may also be beneficial for a controlled and constant release of the drug by diffusion through the pores. Porosity increases the particle surface which is in immediate contact with the release medium. A high level of internal porosity generates a larger inner surface, which can potentially increase the uptake of the release medium into the particles and contribute to drug pore-diffusion.

A precise formulation of the pharmaceutically active compounds is essential to ensure that the microparticles will be deposited to the appropriate part of the lung and deliver the correct amount of pharmaceutically active agent over the appropriate period of time. A careful control over pore formation during particle synthesis and similarly over the so induced porous structure of the microparticles is important to achieve the desired aerodynamic properties together with a favorable kinetic drug release behavior. The development of an appropriate carrier system with adequate aerodynamic properties and evasion of macrophage uptake, that will allow particles to be respirable, yet confer sustained release of drug once deposited in the lung, is therefore a difficult technical problem for the person skilled in the art. The full optimization of such a delivery system with optimal particle properties, such as efficient drug encapsulation, suitable aerodynamics and a predictable, sustained release of the drug is therefore highly challenging for the pharmaceutical technologist.

Large porous particles (LPPs) based on biocompatible and biodegradable poly (lactide-co-glycolide) acid (PLGA), already being used for implantable or injectable depot systems, were suggested as potential sustained-release carriers for pulmonary drug delivery. In the initial studies, sustained serum insulin levels for 4 days were observed following pulmonary delivery in rats. [Edwards D. A., Abdelaziz B.-J., Langer R., J. Appl. Physiol. 85, 379-385 (1998)]. So far, most of the therapeutic agents which were loaded onto inhalable PLGA-LPPs were macromolecular drugs, to achieve either a systemic effect or a local treatment of chronic lung diseases (e.g., COPD, cystic fibrosis). These are typically large soluble therapeutic agents such as peptides and proteins, where the encapsulation within a polymer carrier also serves to prevent degradation of the sensitive macromolecules both on storage and in vivo and deliver the native macromolecule in a sustained manner. Such microparticles have mostly been produced via double emulsion (wᵢ/o/wₑ) extraction methods, which means that the water-soluble drug is first dissolved in an aqueous phase, which is then emulsified with the PLGA polymer solution, prior to further emulsifying with an aqueous solution containing emulsifier (e.g. polyvinyl alcohol (PVA) etc.).

Studies have focused on the preparation of PLGA-based LPPs and especially on strategies how to control particle properties, improve in vivo efficacy and achieve a sustained release of the encapsulated drugs [Ungaro F., D'Angelo I., Miro A., La Rotonda M. I., Quaglia F., J. Pharm. Pharmacol. 64, 1217-1235 (2012)]. Several recent publications evaluate the use of different pore-forming agents (porogens) during the emulsification process to introduce the desired porosity into the particles. Most of these studies used a double emulsion manufacturing process in which various osmotic agents have been employed, such as sodium chloride [Kim H., Lee J., Kim T. H., Lee E. S., Oh K. T., Lee D. H., Park E.-S., Bae Y. H., Lee K. C., Youn Y. S. Pharm. Res. 28 2008-2019 (2011)], albumins [Lee J., Oh Y. J., S. K. Lee, K. Y. Lee, J. Control. Release 146, 61-67 (2010)] and cyclodextrin derivatives [Ungaro F., De Rosa G., Miro A., and Quaglia F., EJPS 28, 423-432 2006; Ungaro F., d'Emmanuele di Villa Bianca R., Giovino C., Miro A., Sorrentino F., Quaglia F., and La Rotonda M. I., J. Control. Release 135, 25-34 (2009)], which cause water influx under an osmotic gradient from the external (wₑ) to the internal (wᵢ) water phase during solvent evaporation (particle hardening), which will then lead to the porous structure of the particles. However, due to the mass exchange and consequent loss of the soluble drugs during the hardening process a poor control of drug encapsulation efficiency (EE %) and drug release is often observed. Another drawback is remaining residues of potentially toxic porogens (e.g. cyclodextrins, Shao Z., Krishnamoorthy R., Mitra A. K., Pharm. Res. 9 1157-1163 (1992). Besides osmotic agents, which have been mostly used for the production of large porous particles containing proteins and peptides as active pharmaceutical ingredients an alternative pore-forming strategy relies on effervescent agents to obtain encapsulation of small and macromolecules. Here, pore formation depends on effervescence rather than on the diffusional mass exchange. Budesonide-loaded large porous PLGA microparticles have been obtained via a double-emulsion solvent evaporation and using ammonium bicarbonate as a porogen, which decomposes into ammonia and carbon dioxide during emulsification. The produced particles showed a sustained release of budesonide in vitro for 24 h and also an improved therapeutic efficacy in a murine asthma model [Oh Y. J., Lee J., Seo J. Y., Rhim T., Kim S.-H., Yoon H. J., Lee K. Y., J. Control. Release, 150, 56-62 (2011)]. The same method was used to prepare Doxorubicin-loaded highly porous PLGA-based LPPs for the treatment of metastatic lung cancer. These were found to be deposited in the lungs of mice and remain in situ for up to 14 days, also tumor growth in the treated mice was significantly reduced [Kim I., Byeon H. J., Kim T. H., Lee E. S., Oh K. T., Shin B. S., Lee K. C., Youn Y. S, Biomaterials, 33, 5574-5583 (2012)]. A drawback of the use of gas-forming agents however lies in the fact that particle size and pore size are usually coupled and therefore cannot be varied independently. Another pore forming strategy, which has been tried in the past, was to use immicible oils (canola or silicon oil) as a porogenic agent for the preparation of PLGA-LPPs encapsulating ciprofloxacin via a double emulsion process. However, only very low encapsulation efficiencies were achieved with a maximum drug loading of 0.4%. Also, the oils had to be extracted from the particles in a subsequent step using an organic solvent to generate the porous structure [Arnold M. E., Gorman E. M., Schieber L. J., Munson E. J., Berland C., J. Control. Release, 121 (1-2), 100-109 (2007)].

Much less is known on the fabrication and the potential of small molecular drug loaded PLGA-based LPPs for pulmonary drug delivery prepared by single emulsion technology. (e.g. oil-in-water (o/w) single emulsion solvent evaporation technique [Wischke C., Schwendemann S. P., Int. J. Pharm., 364, 298-327 (2008)]). PLGA particles prepared by the single-emulsification method have been reported as being naturally less porous as those made by double emulsification [Edwards D. A., Hames J., Caponetti G., Hrkach J., Abdelaziz B.-J., Eskew M. L., Mintzes J., Deaver D., Lotan N., Langer R., Science, 276, 1868-1871 (1997)]. Still less reports on the introduction of porosity and the use of porogenic agents for the manufacture of porous microspheres by a single emulsion method are available.

Extractable porogens, such as poloxamers and fatty acid salts, have been used in combination with a single emulsion protocol for the preparation of porous PLGA microparticles, where the porous structure of the particles is generated by the time difference between PLGA droplet hardening and in situ extraction of the water-soluble porogens from the oil-phase into the water phase. Few reports describe the use of poloxamer copolymers (Pluronics® F68 and F127) as extractable porogens for the preparation of PLGA microparticles, though these were mostly focused on injectable microspheres and thereby implying different requirements regarding the particle properties and the drug loading [Chung H. J., Kim H. K., Yoon J. J., Park T. G, Pharmaceut. Res., 23(8), 1835-1841 (2006); Kim H. K., Chung H. J., Park T. G, 112, 167-174 (2006)]. In a single reference the preparation of PLGA-LPPs for pulmonary drug delivery using a mixture of Pluronics® F68/F127 was reported [Kim H., Park H., Lee J., Kim T. H., Lee E. S., Oh K. T., Lee K. C., Youn Y. S., Biomaterials, 32, 1685-1693 (2011)]. However, in all of the before listed protocols the particles were prepared in a first step and the active agents were then adsorbed/immobilized onto the particles in a second step. In the latter reference the drug (exendin-4) was furthermore acylated with palmitic acid to improve adsorption to the preformed LPPs, while a maximum loading of 5% was achieved. The loaded LPPs were then tested in a murine in vivo model for diabetic inhalation treatment, where a sustained release of the drug was found, which was also attributed to an enhanced hydrophobic interaction with the polymer matrix through the acylated fatty acid residue.

Despite the progress described in the art, there remains a need for improved medicines for pulmonary drug delivery. In particular, there remains a need for respirable pharmaceutical formulations, which reach the targeted lung regions either to achieve a systemic pharmacological effect or for a local treatment of a lung disease, while being adjustable to release the pharmaceutically active agent in/after a predefined time period, e.g. in a sustained manner. In particular, there remains a need for sustained-release formulations for pulmonary administration comprising drug loaded porous microparticles. Furthermore there are so far no satisfactory solutions for a simple, yet efficient preparation of drug loaded porous microparticles for pulmonary administration, where the porosity of the microparticles may readily be adjusted and controlled during the preparation process. In particular there are yet no satisfactory solutions for a single emulsion-solvent evaporation/extraction based method for the preparation of drug loaded porous microparticles for pulmonary administration, where the method dispenses with a step subsequent to particle preparation, (either for loading the drug, e.g. through adsorption/immobilisation, or for introducing porosity, like for example through extraction of the porogenic agent or by treatment with a supercritical fluid such as carbon dioxide above its critical pressure and temperature). In particular there are yet no satisfactory solutions for a single emulsion-solvent evaporation/extraction based method using extractable porogenic agents, which do not have to be separated in a subsequent step and which do not leave potentially harmful residues in the resulting microparticles. Furthermore there are so far no satisfactory solutions for the preparation of porous microparticles for pulmonary drug delivery encapsulating hydrophobic small molecule drugs, especially if these cannot be formulated through the established double emulsion based protocols and if it is desired to employ a porogenic agent to introduce porosity.

These difficulties may have contributed to the fact that, to our knowledge, no inhalable pharmaceutical formulations with a controlled, resp. sustained release of the pharmaceutically active agents are yet on the market. It would therefore be desirable to find a simple process for the preparation of porous microparticles with favorable aerodynamic properties and high encapsulation efficiency for the encapsulated drugs as well as a controllable release rate of the pharmaceutically active agents and where the process does not have the disadvantages mentioned for the processes of the prior art.

The present invention therefore aims at developing a simple and efficient one-pot process for the preparation of porous microparticles encapsulating pharmaceutically active agents, especially small molecule drugs with a low solubility in water and/or organic solvents, where the resulting microparticles are suitable for pulmonary drug delivery and where the process does not have the disadvantages mentioned for the processes of the prior art. The objective was in particular to develop a process, which is flexible in adjusting the aerodynamic properties of the resulting microparticles as well as controlling the in vitro and in vivo release rates of the encapsulated drug. Another objective of the invention was to provide a sustained-release pharmaceutical composition comprising porous microparticles. Moreover, another objective of the invention was to find a suitable porogenic agent, which is compatible with a single emulsion-solvent evaporation/extraction based process and allows for an easy adjustment and fine-tuning of porosity and pore distribution of the prepared microparticles.

The invention pertains to a process for the preparation of porous microparticles for pulmonary drug delivery comprising a matrix material and a pharmaceutically active agent, the process comprising the steps
(i) preparing an o/w emulsion, wherein
   a first phase (a) comprising a pharmaceutically active agent, a matrix material, a porogenic agent and a volatile solvent, is emulsified with
   a second, aqueous phase (b), optionally comprising an emulsifying agent,
(ii) optionally stirring the o/w emulsion resulting from step (i),
(iii) removing the volatile solvent,
(iv) separating the porous microparticles from the remaining phase resulting from step (iii),
(v) optionally drying the porous microparticles resulting from step (iv),
characterized in that the porogenic agent in step (i) is polyvinylpyrrolidone and/or a polyvinylpyrrolidone derivative.

The invention further pertains to porous microparticles for pulmonary drug delivery comprising a matrix material and a pharmaceutically active agent, obtainable by the process according to the invention.

The invention further pertains to a pharmaceutical composition for pulmonary drug delivery comprising porous microparticles comprising a matrix material and a pharmaceutically active agent, obtainable by the process according to the invention.

The invention further pertains to a use of the pharmaceutical composition according to the invention for use in the treatment and/or prevention of diseases, preferably pulmonary diseases or conditions of the lungs and/or airways.

The invention further pertains to the use of polyvinylpyrrolidone and/or a polyvinylpyrrolidone derivative as a porogenic agent for the preparation of porous microparticles for pulmonary drug delivery.

The process according to the invention is a simple and efficient one-pot single-emulsion based method for the preparation of porous microparticles for pulmonary drug delivery. The method is especially suitable for the encapsulation of hydrophobic small molecule drugs and achieves good encapsulation efficiency of the pharmaceutically active agents. A further advantage of the process according to the invention is that the process can be conducted in one step without the need to subsequently load the microparticles with the pharmaceutically active agent or to separate the porogenic agent in an extra step. It is not necessary to wash out possible residues of the porogenic agent, due to the good biocompatibility of the polyvinylpyrrolidone employed. The porosity and the physicochemical properties of the microparticles can favorably be adjusted by the amount of polyvinylpyrrolidone employed. The microparticles have favorable properties, such as an ideal MMAD that allows for lung deposition, large geometric diameter which prevents the particle from macrophage uptake and slows down mucociliary clearance, sufficient drug loading capacity, exhibition of sustained drug release, as well as reproducible particle morphology. A further advantage of the porous microparticles produced according to the invention is that these may be administered in form of a pulmonary sustained-release formulation, where the release rate may be controlled via the matrix type as well as the porogen type and amount used during the preparation process.

The invention is illustrated in detail hereinafter. Various embodiments can be combined here with one another as desired, unless the opposite is apparent to the person skilled in the art from the context.

As used herein the terms “comprise”, “comprising”, “include” and “including” are intended to be open, non-limiting terms, unless the contrary is expressly indicated.

The use of the term “about” to qualify a numerical range, qualifies all numbers within the range, unless the context indicates otherwise.

### Porogenic Agent

In the sense of the invention polyvinylpyrrolidone and/or a polyvinylpyrrolidone derivative is employed as an extractable porogenic agent within the single emulsion based process according to the invention. It may be referred to herein generally as “porogen”, “porogenic agent”, “pore forming agent” or the like.

Polyvinylpyrrolidones (povidones, PVP, Kollidon®, Plasdone®) are commercially available hydrophilic polymers suitable for use in solid pharmaceutical preparations. They are polydisperse macromolecular molecules, with a chemical name of 1-ethenyl-2-pyrrolidinone polymers and 1-vinyl-2-pyrrolidinone polymers. Povidone polymers are produced commercially as a series of products having mean molecular weights ranging from about 2000-3000 (e.g. PVP K-12) to about 3000000 (e.g. K-120) daltons. Various types of PVP are commercially available (e.g., soluble grades: povidone, insoluble grades: crospovidone) and have been used in the pharmaceutical industry for several applications. Soluble PVP grades are for example employed as binders, solubilisation enhancers, film formers, taste masking agents, lyophilisation agents, suspending agents, hydrophilizers, adhesives and other.

Polyvinylpyrrolidones have been incorporated in tablets and microspheres to enhance or extend the release of the pharmaceuticals. EP 2361616 A1 and Verma R. K., Kaushal A. M., Garg S., Int. J. Pharm., 263, 9-24 (2003) disclose coated solid dosage forms, preferably tablets, where the coating may comprise PVP as a hydrophilic pore former. In contact with water PVP will dissolve and thus generate water-filled channels, which will support dissolving of the tablet and result in faster drug release. Povidone K-30 has been used as a channeling agent in injectable microspheres of poly (lactic acid) for long-acting controlled-release parenteral administration. It was found that release of encapsulated drug, as well as the drug content, depended on the amount of PVP used. The microspheres also showed visible pores on the surface [Lalla J. K., Sapna K., J. Microencapsul., 10, 449-460 (1993)]. In CN101536984A polyvinylpyrrolidone has been listed as potential pore forming agent for manufacturing of microspheres for intravenous application via a double emulsion technology. These microspheres contain the hydrophilic peptide endostatin as active ingredient. PVP has also been described as an extractable porogen for chitosan-based membranes [Zeng M., Fang Z., Xu C., J. Membr. Sci., 230, 175-181 (2004)] and porous microspheres [Zeng M., Zhang X., Qi C., Zhang X.-M., Int. J. Biol. Macromol., 51, 730-737 (2012)], where the PVP was extracted in a subsequent step in hot aqueous solution. It was found that the microspheres however still contained considerable amount of PVP polymer due to strong interactions with the chitosan matrix. Polyvinylpyrrolidone may also be used as matrix material/excipient for (porous) microparticles, as reported in WO 07/086039 A1 or WO 05/027875 A1. WO 06/088894 A2 discloses benzodiazepine nanoparticles for injection or inhalation, which comprise povidone polymers as a surface stabilizer.

Povidone polymers are prepared by, for example, Reppe's process, comprising: (a) obtaining 1,4-butanediol from acetylene and formaldehyde by the Reppe butadiene synthesis; (b) dehydrogenating the 1,4-butanediol over copper at 200° C. to form γ-butyrolactone; and (c) reacting γ-butyrolactone with ammonia to yield pyrrolidone. Subsequent treatment with acetylene gives the vinyl pyrrolidone monomer. Polymerization is carried out by heating in the presence of water and ammonia. The manufacturing process for povidone polymers produces polymers containing molecules of unequal chain length, and thus different molecular weights. The molecular weights of the molecules vary about a mean or average for each particular commercially available grade. Because it is difficult to determine the polymer's molecular weight directly, the most widely used method of classifying various molecular weight grades is by K-values, based on viscosity measurements. The K-values of various grades of povidone polymers represent a function of the average molecular weight, and are derived from viscosity measurements and calculated according to Fikentscher's formula. The weight-average of the molecular weight, M_{w}, is determined by methods that measure the weights of the individual molecules, such as by light scattering. If in doubt, the data on the K value from the European Pharmacopeia (Ph. Eur.) are used.

Table 1 provides molecular weight data for several commercially available povidone polymers, all of which are soluble.

| | | Mᵥ | M_{w} | Mₙ |
|---|---|---|---|---|
| Povidone | K-Value | (Daltons)* | (Daltons)* | (Daltons)* |
| Plasdone C-15 ® | 17 ± 1 | 7000 | 10500 | 3000 |
| Plasdone C-30 ® | 30.5 ± 1 | 38000 | 62500 | 16500 |
| Kollidon 12 PF ® | 11-14 | 3900 | 2000-3000 | 1300 |
| Kollidon 17 PF ® | 16-18 | 9300 | 7000-11000 | 2500 |
| Kollidon 25 ® | 24-32 | 25700 | 28000-34000 | 6000 |

*Mᵥ is the viscosity-average molecular weight, Mₙ is the number-average molecular weight, and M_{w} is the weight-average molecular weight. M_{w} and Mₙ were determined by light-scattering and ultra-centrifugation, and Mᵥ was determined by viscosity measurements.

The polyvinylpyrrolidone (derivative) employed in the sense of the present invention preferably has a good solubility in water. In this case, the polyvinylpyrrolidone (derivative) is normally linear and not crosslinked. These polyvinylpyrrolidones also have a very good solubility in various solvents, which extends from extremely hydrophilic solvents, such as water (>100 mg/ml), to more hydrophobic liquids, such as butanol or methylene chloride (>100 mg/ml).

The polyvinylpyrrolidone (derivative) employed normally has a K value of at least 12. The polyvinylpyrrolidone (derivative) which is used in the process according to the invention normally has a K value of from 12 to 120, preferably from 12 to 40, particularly preferably from 12 to 17. In a preferred embodiment of the invention polyvinylpyrrolidone with a K value of 12 is used.

The polyvinylpyrrolidone (derivative) is normally used as porogenic agent in an amount of e.g. 1% up to about 200%, by weight (w/w) relative to the matrix material employed in the process according to the invention. In an embodiment of the inventive process, the polyvinylpyrrolidone (derivative) is used in a ratio of from 5% to 100%, preferably of from 5% to 50%, more preferably of from 10% to 30%, particularly preferably of from 15% to 25% by weight (w/w), relative to the matrix material.

When desirable, further porogenic agents, such as those listed before, may be used in addition to polyvinylpyrrolidone (derivatives) and combined with the process according to the invention. These will be chosen to show good compatibility with the single emulsion process according to the present invention. In a preferred embodiment of the process according to the invention no additional porogenic agents are used. Preferably polyvinylpyrrolidone is used as the sole porogenic agent.

It has been found that polyvinylpyrrolidone can favorably be used as a porogenic agent in a single emulsion-solvent evaporation process. The novel method can be conducted as a one-pot, one-step process under efficient encapsulation of the active agent. Surprisingly, the polyvinylpyrrolidone (derivative) can be used as an extractable porogenic agent during the inventive single emulsion process, where the extraction of the porogenic agent from the formed drug-loaded microparticles takes place simultaneously to particle formation/hardening. Still, significant drug loss through mass exchange does not take place, so that good encapsulation efficiency for the active agents is achieved. Previously reported methods with extractable porogens needed a subsequent step to their preparation either for drug loading, extraction of the porogen or modification of the pore structure. Advantageously, even if residual amounts of polyvinylpyrrolidone remain within the formed microparticles, these do not have to be removed in an extra step, due to the established biocompatibility of polyvinylpyrrolidone. The produced microparticles have favorable aerodynamic properties for pulmonary administration and also show good porosity with a regular pore distribution, which may be controlled by the amount and ratio of the porogenic agent employed. Surprisingly, the microparticles thus obtained also possess improved particle morphology/reduced aggregation tendency when compared with those obtained when applying alternative extractable porogenic agents with a similar physicochemical profile.

If it is desired to remove any remaining polyvinylpyrrolidone from the microparticles (e.g. to further decrease their density), such a treatment may be done by an additional washing step, preferably with water. For a complete extraction of residues of the porogenic agent from the inner phase of the microparticles it may be necessary to repeat such an extraction several times. In a preferred embodiment of the process according to the invention, remaining residues of the polyvinylpyrrolidone are not removed, e.g. by extraction, from the porous microparticles before these are furnished for pulmonary administration. In one particular embodiment according to the invention, the porous microparticles comprising the pharmaceutically active agent are not further purified subsequent to their preparation and before administration in form of a pharmaceutical composition.

### Pharmaceutically Active Agent

The “pharmaceutically active agent” is a therapeutic, diagnostic, or prophylactic agent. It may be referred to herein generally as a “drug”, “active agent” or “pharmaceutically active ingredient (API)”. As used herein, the terms further include any physiologically or pharmacologically active substance that produces a localized or systemic effect in a patient.

Suitable pharmaceutically active agents are in principle all pharmaceutically active chemical compounds, which show compatibility with the employed single emulsion process according to the invention. The identity of the active agent may therefore be limited by its solubility or partition coefficient between the organic and aqueous emulsion phases. If the solubility in the aqueous phase is too high some of the drug may be lost during emulsification and/or particle hardening resulting in a lower efficiency for drug encapsulation.

The log P value represents a measure for the lipophilicity of a chemical entity (e.g. an active pharmaceutical ingredient), where P (partition coefficient) is the ratio of the concentration of a chemical entity (measured at a pH value where the chemical entity is in an unionized form) in a mixture of two inmiscible phases at equilibrium, usually between octanol and water (herein mentioned log P values refer to octanol/water partition coefficients).

$$log {P}_{\frac{oct}{wat}} = log ( \frac{[ solute ] \frac{un \text{-} ionized}{octanol}}{[ solute ] \frac{un \text{-} ionized}{water}} ) \qquad ( EQ . 1 )$$

Alternatively, log P can be experimentally determined using high performance liquid chromatography (HPLC) by correlating the chemical entity's retention time with similar compounds with known log P values (Valko K, J. Chromatogr. A., 28, 299-310 (2004). Furthermore, several in silico tools to predict log P (so-called computational log P=clogP) have been developed and are commonly used within pharmaceutical sciences (Mannhold R., Poda G. I., Ostermann C., Tetko I. V., J. Pharm. Sci. 98, 861-893 (2009). The active agent preferably has a log P_{oct/wat} value of from −1.0 to +7.

In a preferred embodiment according to the invention, the pharmaceutically active agent is a (hydrophobic) small molecule compound with low aqueous solubility. For those compounds only a few specific protocols for the encapsulation in porous microparticles have been reported so far. As used herein, the term “low aqueous solubility” means that the drug has a solubility of less than 1 mg/mL, and preferably less than 0.1 g/mL, in aqueous media at 15-25° C. and physiologically neutral pH (about 5.0-8.0), e.g. a very slightly soluble (0.1 mg/ml-1 mg/ml) or practically insoluble (<0.1 mg/ml) drug (according to the solubility definitions in Pharm. Eur. 8-5, Chapter 1.4, Solubility).

In a preferred embodiment according to the invention, the pharmaceutically active agent exhibits sufficient solubility (>10 mg/ml, preferably >30 mg/ml, preferably >100 mg/ml) in a water-immiscible organic phase, preferably an organic solvent of class 2 or class 3 (according to The International Conference on Harmonisation of Technical Requirements for Registration of Pharmaceuticals for Human Use (ICH) guideline Q3C; www.ich.org) or a mixture thereof, preferably in a water-immiscible organic class 2 or 3 solvent chosen from the list of the following solvents: dichloromethane, cyclohexane, hexane, methylbutylketone, N-methylpyrrolidone, tert.-butylmethylether, ethylacetate, diethylether, heptane, pentane or mixtures thereof, especially preferably in dichloromethane and N-methylpyrrolidone or a mixture thereof.

A variety of pharmaceutically active agents may be employed in the process according to the invention and in the pharmaceutical compositions. Representative examples of suitable pharmaceutically active agents include the following categories and examples of pharmaceutically active agents and alternative forms of these pharmaceutically active agents such as alternative salt forms, free acid forms, free base forms, and hydrates:

As suitable active compounds, we may mention for example and preferably:
organic nitrates and NO-donors, for example sodium nitroprusside, nitroglycerin, isosorbide mononitrate, isosorbide dinitrate, molsidomine or SIN-1, and inhalational NO;
compounds that inhibit the degradation of cyclic guanosine monophosphate (cGMP) and/or cyclic adenosine monophosphate (cAMP), for example inhibitors of phosphodiesterases (PDE) 1, 2, 3, 4 and/or 5, in particular PDE 4 inhibitors such as roflumilast or revamilast and PDE 5 inhibitors such as sildenafil, vardenafil, tadalafil, udenafil, dasantafil, avanafil, mirodenafil or lodenafil;
NO- and haem-independent activators of guanylate cyclase, in particular the compounds described in WO 01/19355, WO 01/19776, WO 01/19778, WO 01/19780, WO 02/070462, WO 02/070510 and WO2014/012934;
NO-independent but haem-dependent stimulators of guanylate cyclase, in particular riociguat and the compounds described in WO 00/06568, WO 00/06569, WO 02/42301, WO 03/095451, WO 2011/147809, WO 2012/004258, WO 2012/028647 and WO 2012/059549;
sGC stimulators and sGC activator compounds described in WO 03/097063, WO 03/09545, WO 04/009589, WO 03/004503, WO 02/070462, WO 2007/045366, WO 2007/045369, WO 2007/045370, WO 2007/045433, WO 2007/045367, WO 2007/124854, WO 2007/128454, WO 2008/031513, WO 2008/061657, WO 2008/119457, WO 2008/119458, WO 2009/127338, WO 2010/079120, WO 2010/102717, WO 2011/051165, WO 2011/147809, WO 2011/141409, WO 2014/012935, WO 2012/059549, WO 2012/004259, WO 2012/004258, WO 2012/059548, WO 2012/028647, WO 2012/152630, WO 2012/076466, WO 2014/068099, WO 2014/068104, WO 2012/143510, WO 2012/139888, WO 2012/152629, WO 2013/004785, WO 2013/104598, WO 2013/104597, WO 2013/030288, WO 2013/104703, WO 2013/131923, WO 2013/174736, WO 2014/012934, WO 2014/068095, WO 2014/195333, WO 2014/128109, WO 2014/131760, WO 2014/131741, WO 2015/018808, WO 2015/004105, WO 2015/018814, WO 98/16223, WO 98/16507, WO 98/23619, WO 400/06569, WO 01/19776, WO 01/19780, WO 01/19778, WO 02/042299, WO 02/092596, WO 02/042300, WO 02/042301, WO 02/036120, WO 02/042302, WO 02/070459, WO 02/070460, WO 02/070461, WO 02/070510, WO 2012/165399, WO 2014/084312, WO 2011115804, WO 2012003405, WO 2012064559, WO 2014/047111, WO 2014/047325, WO 2011/149921, WO 2010/065275, WO 2011/119518
prostacyclin analogs and IP receptor agonists, for example and preferably iloprost, beraprost, treprostinil, epoprostenol or NS-304;
endothelin receptor antagonists, for example and preferably bosentan, darusentan, ambrisentan or sitaxsentan;
human neutrophile elastase (FINE) inhibitors, for example and preferably sivelestat or DX-890 (Reltran);
compounds which inhibit the signal transduction cascade, in particular from the group of the tyrosine kinase inhibitors, for example and preferably dasatinib, nilotinib, bosutinib, regora-fenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib, masitinib or tandutinib;
Rho kinase inhibitors, for example and preferably fasudil, Y-27632, SLx-2119, BF-66851, BF-66852, BF-66853, KI-23095 or BA-1049;
anti-obstructive agents as used, for example, for the therapy of chronic-obstructive pulmonary disease (COPD) or bronchial asthma, for example and preferably inhalatively or systemically administered beta-receptor mimetics (e.g. bedoradrine) or inhalatively administered anti-muscarinergic substances;
antiinflammatory and/or immunosuppressive agents as used, for example for the therapy of chronic-obstructive pulmonary disease (COPD), of bronchial asthma or pulmonary fibrosis, for example and preferably systemically or inhalatively administered corticosteroides, flutiform, pirfenidone, acetylcysteine, budesonide, azathioprine or BIBF-1120;
antibacterial, antiprotozoal, antimucosal, antiparasitic, and antiviral agents;
chemotherapeutics as used, for example, for the therapy of neoplasias of the lung or other organs;
active compounds used for the systemic and/or inhalative treatment of pulmonary disorders, for example for cystic fibrosis (alpha-1-antitrypsin, aztreonam, ivacaftor, lumacaftor, ataluren, amikacin, levofloxacin), chronic obstructive pulmonary diseases (COPD) (LAS40464, PT003, SUN-101), acute respiratory distress syndrome (ARDS) and acute lung injury (ALI) (interferon-beta-1a, traumakines), obstructive sleep apnoe (VI-0521), bronchiectasis (mannitol, ciprofloxacin), Bronchiolitis obliterans (cyclosporine, aztreonam) and sepsis (pagibaximab, Voluven, ART-123);
active compounds used for treating muscular dystrophy, for example idebenone;
antithrombotic agents, for example and preferably from the group of platelet aggregation inhibitors, anticoagulants or profibrinolytic substances;
active compounds for lowering blood pressure, for example and preferably from the group of calcium antagonists, angiotensin AII antagonists, ACE inhibitors, endothelin antagonists, renin inhibitors, alpha-blockers, beta-blockers, mineralocorticoid receptor antagonists and diuretics; and/or
active compounds that alter fat metabolism, for example and preferably from the group of thyroid receptor agonists, cholesterol synthesis inhibitors such as for example and preferably HMG-CoA-reductase or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbers, bile acid reabsorption inhibitors and lipoprotein(a) antagonists.

Antithrombotic agents are preferably to be understood as compounds from the group of platelet aggregation inhibitors, anticoagulants or profibrinolytic substances.

In a particular aspect of the invention an active compound is a platelet aggregation inhibitor, for example and preferably aspirin, clopidogrel, ticlopidine or dipyridamole.

In a particular aspect of the invention an active compound is a thrombin inhibitor, for example and preferably ximelagatran, melagatran, dabigatran, bivalirudin or Clexane.

In a particular aspect of the invention an active compound is a GPIIb/IIIa antagonist, for example and preferably tirofiban or abciximab.

In a particular aspect of the invention an active compound is a factor Xa inhibitor, for example and preferably rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, an active compound is heparin or a low molecular weight (LMW) heparin derivative.

In a particular aspect of the invention an active compound is a vitamin K antagonist, for example and preferably coumarin.

The agents for lowering blood pressure are preferably to be understood as compounds from the group of calcium antagonists, angiotensin AII antagonists, ACE inhibitors, endothelin antagonists, renin inhibitors, alpha-blockers, beta-blockers, mineralocorticoid-receptor antagonists and diuretics.

In a particular aspect of the invention an active compound is a calcium antagonist, for example and preferably nifedipine, amlodipine, verapamil or diltiazem.

In a preferred embodiment of the invention, an active compound is an alpha-1-receptor blocker, for example and preferably prazosin.

In a particular aspect of the invention an active compound is a beta-blocker, for example and preferably propranolol, atenolol, timolol, pindolol, alprenolol, oxprenolol, penbutolol, bupranolol, metipranolol, nadolol, mepindolol, carazolol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, an active compound is an angiotensin AII antagonist, for example and preferably losartan, candesartan, valsartan, telmisartan or embursatan or a dual angiotensin AII antagonist/neprilysin-inhibitor, by way of example and with preference LCZ696 (valsartan/sacubitril).

In a preferred embodiment of the invention, an active compound is an ACE inhibitor, for example and preferably enalapril, captopril, lisinopril, ramipril, delapril, fosinopril, quinopril, perindopril or trandopril.

In a preferred embodiment of the invention, an active compound is an endothelin antagonist, for example and preferably bosentan, darusentan, ambrisentan or sitaxsentan.

In a particular aspect of the invention an active compound is a renin inhibitor, for example and preferably aliskiren, SPP-600 or SPP-800.

In a particular aspect of the invention an active compound is a mineralocorticoid-receptor antagonist, for example and preferably spironolactone, eplerenone and finerenone (BAY 94-882).

In a particular aspect of the invention an active compound is a diuretic, for example and preferably furosemide, bumetanide, Torsemide, bendroflumethiazide, chlorthiazide, hydrochlorthiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Agents altering fat metabolism are preferably to be understood as compounds from the group of CETP inhibitors, thyroid receptor agonists, cholesterol synthesis inhibitors such as HMG-CoA-reductase or squalene synthesis inhibitors, the ACAT inhibitors, MTP inhibitors, PPAR-alpha, PPAR-gamma and/or PPAR-delta agonists, cholesterol-absorption inhibitors, polymeric bile acid adsorbers, bile acid reabsorption inhibitors, lipase inhibitors and the lipoprotein(a) antagonists.

In a particular aspect of the invention an active compound is a CETP inhibitor, for example and preferably torcetrapib, (CP-5294/4), JJT-705 or CETP-vaccine (Avant).

In a particular aspect of the invention an active compound is a thyroid receptor agonist, for example and preferably D-thyroxin, 3,5,3′-triiodothyronin (T3), CGS 23425 or axitirome (CGS 26214).

In a preferred embodiment of the invention, an active compound is an HMG-CoA-reductase inhibitor from the class of statins, for example and preferably lovastatin, simvastatin, pravastatin, fluvastatin, atorvastatin, rosuvastatin or pitavastatin.

In a particular aspect of the invention an active compound is a squalene synthesis inhibitor, for example and preferably BMS-188494 or TAK-475.

In a preferred embodiment of the invention, an active compound is an ACAT inhibitor, for example and preferably avasimibe, melinamide, pactimibe, eflucimibe or SMP-797.

In a preferred embodiment of the invention, an active compound is an MTP inhibitor, for example and preferably implitapide, BMS-201038, R-103757 or JTT-130.

In a particular aspect of the invention an active compound is a PPAR-gamma agonist, for example and preferably pioglitazone or rosiglitazone.

In a particular aspect of the invention an active compound is a PPAR-delta agonist, for example and preferably GW 501516 or BAY 68-5042.

In a particular aspect of the invention an active compound is a cholesterol-absorption inhibitor, for example and preferably ezetimibe, tiqueside or pamaqueside.

In a particular aspect of the invention an active compound is a lipase inhibitor, for example and preferably orlistat.

In a particular aspect of the invention an active compound is a polymeric bile acid adsorber, for example and preferably cholestyramine, colestipol, colesolvam, CholestaGel or colestimide.

In a particular aspect of the invention an active compound is a bile acid reabsorption inhibitor, for example and preferably ASBT (=IBAT) inhibitors, e.g. AZD-7806, S-8921, AK-105, BARI-1741, SC-435 or SC-635.

In a particular aspect of the invention an active compound is a lipoprotein(a) antagonist, for example and preferably gemcabene calcium (CI-1027) or nicotinic acid.

A preferred group of pharmaceutically active agents for the treatment of pulmonary hypertension are sGC activators, for example and preferably cinaciguat (BAY 58-2667).

Cinaciguat is practically insoluble in water and some commonly used organic solvents such as dichloromethane, acetonitrile, only very slightly soluble in ethanol, methanol, ethyl acetate, and slightly soluble in acetone. Cinaciguat has been encapsulated in non-porous, dipalmitoyl-phosphatidylcholine/albumin/lactose (DAL)-based microparticles with a size range of from 2 to 6 μm. These were obtained via a spray-drying method and were tested for inhalation in an awake lamb model of acute pulmonary hypertension [Evgenov O. V., Kohane D. S, Bloch K. D., Stasch J.-P., Volpato G. P., Bellas E., Evgenov N. V., Buys E. S., Gnoth M. J., Graveline A. R., Liu R., Hess D. R., Langer R., Zapol W. M., Am. J. Respir. Crit. Care Med., 176, 1138-1145 (2007)]. The pulmonary vasodilation that occurred after inhaling DAL-cinaciguat microparticles was dose dependent and lasted for up to 120 min.

### Matrix Material

Suitable matrix materials are those, which are compatible with the employed single emulsion method. In general these are matrix materials, which exhibit sufficient solubility (>10 mg/ml, preferably >30 mg/ml, more preferably >100 mg/ml) in the organic solvent/solvent mixture that is being used for the preparation procedure.

In a preferred embodiment according to the invention, the matrix material is a biocompatible, preferably biodegradable, polymer. As used herein, the term “biocompatible” describes a material which may be inserted, e.g. by inhalation, into a living subject without causing an adverse response. For example, it does not cause inflammation or acute rejection by the immune system that cannot be adequately controlled. It will be recognized that “biocompatible” is a relative term, and some degree of immune response is to be expected even for substances that are highly compatible with living tissue. An in vitro test to assess the biocompatibility of a substance is to expose it to cells; biocompatible substances will typically not result in significant cell death (for example, >20%) at moderate concentrations (for example, 50 μg/10⁶ cells). As used herein, the term “biodegradable” describes a polymeric matrix material which degrades in a physiological environment to form monomers and/or other non-polymeric moieties that can be reused by cells or disposed of without significant toxic effect. Degradation may be biological, for example, by enzymatic activity or cellular machinery, or may be chemical (e.g. hydrolysis). Degradation of a polymer may occur at varying rates, with a half-life in the order of days, weeks, months, or years, depending on the polymer or copolymer used.

The biocompatible and/or biodegradable polymer can be a poly(lactide), a poly(glycolide), a poly-(lactide-co-glycolide) (PLGA), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyano-acrylate, a poly(oxyethylene)/poly(oxypropylene) copolymer, polyacetals, polyketals, poly-phosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof. Preferably the matrix polymer is selected from the group consisting of poly(lactide-co-glycolide), poly(lactide), or poly(glycolide) and derivatives thereof.

In specific aspects, the biocompatible or biodegradable polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. In addition to copolymers comprising glycolide and DL-lactide or L-lactide, copolymers of L-lactide and DL-lactide are commercially available. Homo-polymers of lactide or glycolide are also commercially available. When the biodegradable and/or biocompatible polymer is poly(lactide-co-glycolide), poly(lactide), or poly(glycolide), the amount of lactide and glycolide in the polymer can vary. In a further aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 0 to 50 mole %, 0 to 40 mole %, 0 to 30 mole %, or 0 to 20 mole % glycolide, preferably 40 to 60 mole % lactide and 40 to 60 mole % glycolide, preferably 48 to 52 mole % lactide and 48 to 52 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), 52:48 poly(lactide-co-glycolide), 48:52 poly(lactide-co-glycolide), or 50:50 poly(lactide-co-glycolide), where the ratios are mole ratios. When the biodegradable and/or biocompatible polymer is poly(lactide-co-glycolide), the inherent viscosity (measured at 0.1% polymer in CHCl₃ (w/v) at 25° C. using an Ubbelhode size OC glass capillary viscometer) is from 0.05 to 1.0 dL/g, preferably from 0.1 to 0.5 dL/g, more preferably from 0.16 to 0.44 dL/g. The biodegradable polymer may either be an end-capped polymer (terminal carboxy-groups are esterified) or comprise mainly free terminal carboxy groups (acid). Preferably, the biodegradable polymer comprises mainly free terminal carboxy groups. In a preferred embodiment the matrix material is poly(lactide-co-glycolide) acid (PLGA). The biodegradable and/or biocompatible polymer can also be a poly(caprolactone) or a poly(lactide-co-caprolactone). The polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

Table 2 provides molecular weight data for several commercially available PLGA polymers, which may be typically and preferably employed as matrix material in the inventive process.

| | Composition | | Inherent | |
|---|---|---|---|---|
| | (D,L-lactide: | Acid | viscosity (0.1% | Molecular |
| | glycolide; | number | in chloroform, | weight |
| PLGA type | molar ratio) | [mg KOH/g] | 25° C.) | [Da] |
| RESOMER ® RG 502* | 48:52 to 52:48 | ≤1 mg | 0.16-0.24 dl/g | 7000-17000 |
| RESOMER ® RG 502H** | 48:52 to 52:48 | ≥6 mg | 0.16-0.24 dl/g | 7000-17000 |
| RESOMER ® RG 503* | 48:52 to 52:48 | ≤1 mg | 0.32-0.44 dl/g | 24000-38000 |
| RESOMER ® RG 503H** | 48:52 to 52:48 | ≥3 mg | 0.32-0.44 dl/g | 24000-38000 |

*ester-terminated,
**acid-terminated.

Especially preferred for the inventive process is PLGA 503H as matrix polymer.

It is understood that any combination of the aforementioned biodegradable polymers can be used, including, but not limited to, copolymers thereof, mixtures thereof, or blends thereof. Likewise, it is understood that when a residue of a biodegradable polymer is disclosed, any suitable polymer, copolymer, mixture, or blend, that comprises the disclosed residue, is also considered disclosed. When multiple residues are individually disclosed (i.e., not in combination with another), it is understood that any combination of the individual residues can be used.

### Process for Manufacturing

The process for the preparation of the porous microparticles according to the invention corresponds to analogous single emulsion-solvent evaporation methods described in the state of the art [Rosca R. D., Watari F., Uo M., J Control. Release, 99, 271-280 (2004); Li M., Rouaud O., Poncelet D., Int. J. Pharm., 363, 26-39 (2008); Wischke C., Schwendeman S. P. Int. J. Pharm., 364, 298-327 (2008)].

An emulsification-solvent evaporation process in general constitutes of two fundamental steps: (1) The emulsification of a polymer solution comprising the pharmaceutically active agent, followed by (2) particle hardening through solvent evaporation and polymer precipitation.

The process according to the invention comprises the following steps:
(i) preparing an o/w emulsion, wherein
   a first phase (a) comprising a pharmaceutically active agent, a matrix material, a porogenic agent and a volatile solvent, is emulsified with
   a second, aqueous phase (b), optionally comprising an emulsifying agent,
(ii) optionally stirring the o/w emulsion resulting from step (i),
(iii) removing the volatile solvent,
(iv) separating the porous microparticles from the remaining phase resulting from step (iii),
(v) optionally drying the porous microparticles resulting from step (iv),
wherein polyvinylpyrrolidone and/or a polyvinylpyrrolidone derivative is used as the porogenic agent in step (i).

### Step (i):

The first phase (a) (“organic phase”, “oil phase”) comprises the matrix material, the pharmaceutically active agent and the porogenic agent dissolved or dispersed in a suitable volume of a solvent and can be provided using any suitable means (e.g. stirring, mixing means). Suitable solvents are those, which show good compatibility with the employed single emulsion method. A solvent can be selected based on its biocompatibility as well as the solubility or dispersability of the matrix material, the porogenic agent and/or the pharmaceutically active agent. For example, the ease with which the matrix material is dissolved in the solvent and the lack of detrimental effects of the solvent on the pharmaceutically active agent to be delivered are factors to consider in selecting the solvent. Additionally, the solvent can be selected based on its immiscibility with the aqueous phase. Organic solvents will typically be used to dissolve hydrophobic and some hydrophilic matrix materials. Thus, a wide variety of organic solvents can be used. Preferably the organic solvent/solvent mix is volatile, which means it has a low enough boiling point that the solvent can be removed under atmospheric pressure or under vacuum. Preferred solvents are acceptable for administration to humans in a trace amount (e.g. <50 mg/day/human).

Preferably the solvent or solvent mix is a water-immiscible solvent or solvent mix, preferably an organic solvent of class 2 and class 3 (according to The International Conference on Harmonisation of Technical Requirements for Registration of Pharmaceuticals for Human Use (ICH) guideline Q3C; www.ich.org) or a mixture thereof, more preferably a water-immiscible organic class 2 or 3 solvent from the list of the following solvents: dichloromethane, cyclohexane, hexane, methylbutylketone, N-methylpyrrolidone, tert-butylmethylether, ethylacetate, diethylether, heptane, pentane or mixtures thereof, particularly preferably dichloromethane and N-methylpyrrolidone or a mixture thereof.

The matrix material can be present in the first phase in any desired weight %. For example, the matrix material can be present in the first phase in about 0.1% to about 60% weight to volume (w/v), including without limitation, about 5%, 10%, 15%, 20%, 30%, 40%, or 50% weight to volume (w/v). In general, the matrix material is dissolved in the solvent to form a matrix material solution having a concentration of between 0.1 and 60% weight to volume (w/v), more preferably between 5% and 30% weight to volume (w/v). In a preferred embodiment according to the invention the matrix material is used in an amount of 15 to 25% weight to volume (w/v). For example, the pharmaceutically active ingredient (API) can be present in the first phase in about 0.01% to about 90% relative to the sum of amounts of matrix and API, weight to weight (w/w), including without limitation, about 0.1%, 0.5%, 1%, 5%, 10%, 15%, 20%, 30%, 40%, 50%, 60%, 70%, or 80% relative to the sum of amounts of matrix and API (w/w). In a preferred embodiment according to the invention the pharmaceutically active agent is used in an amount of 1 to 10% relative to the sum of amounts of matrix and API (w/w). For example, the porogenic agent can be present in the first phase in about 1% up to about 200%, by weight (w/w) relative to the matrix employed in the process according to the invention, including without limitation, about 5%, 10%, 15%, 20%, 30%, 40%, 50%, 60%, 70%, 80%, 100%, 120%, and 150%. In an embodiment of the inventive process, the porogenic agent is used in a ratio of from 5% to 100%, preferably of from 5% to 50%, more preferably of from 10% to 30%, particularly preferably of from 15% to 25%, by weight (w/w), relative to the matrix material.

The first phase can further comprise additives such as cosolvents, surfactants, emulsifiers, blends of two or more polymers, or a combination thereof, among other additives.

The second phase is in form of an aqueous phase. In one aspect, water can be mixed with another water-miscible solvent, which at the same time must not be miscible with the organic solvent used for the preparation of the first phase. For example, methanol may be added to the second phase, in case n-heptane or cyclohexane is used for the preparation of the first phase. In various aspects, the second phase can contain other excipients, such as buffers, salts, sugars, surfactants, emulsifiers, and/or viscosity-modifying agents, or combinations thereof. In a preferred embodiment the aqueous phase further comprises an emulsifying agent. The emulsifying agent may serve to form stable microdroplets with an inner organic solvent phase and an outer aqueous phase, from which during the further process steps (e.g. by stirring and subsequent solvent evaporation) formation of solid porous microparticle of reproducible morphology, size, and aerodynamic diameter occurs.

Non-limiting examples of emulsifying agents include those from the group of anionic emulsifiers (e.g. sodium lauryl sulfate), cationic emulsifiers (e.g. cetyl pyridinium chloride), amphoteric emulsifiers (e.g. lecithin), and non-ionogenic emulsifiers (e.g. macrogol stearate, macrogol sorbitan oleate, polyvinylalcohol). The emulsifying agent is typically used in a concentration of from 0.05% to 5% by weight (w/v) in the aqueous phase. In an embodiment of the inventive process, the aqueous phase further comprises polyvinyl alcohol (PVA) as emulsifying agent in a concentration of from 0.05% to 5%, preferably from 0.1% to 3%, more preferably from 0.1 to 0.5%, by weight (w/v) in the aqueous phase, particularly preferably at a concentration of 0.5% by weight (w/v) in the aqueous phase. PVA is commercially available in various grades. The various PVA types available differ in their degree of hydrolysis; completely (>98 mole-%) hydrolysed, medium (90.5-96.5 mole-%) hydrolysed, partially (˜8-89 mole-%) hydrolysed); and degree of polymerization (usually ˜500-2500 monomers) the latter being reflected in different viscosities. In a preferred embodiment the emulsifier is PVA 205. In another preferred embodiment the emulsifier is PVA 217.

The first phase (a) and the second phase (b) may be used in any ratio, which provides a stable emulsion after emulsification. The ratios typically employed in the inventive process range between 1/20 and 1/600 by volume (v/v) [phase (a)/phase (b)]. In a preferred embodiment the ratio between phase (a) and phase (b) is in the range from 3/300 (v/v) to 3/500 (v/v).

According to the present invention, the first phase (a) and the second phase (b) are subjected to an emulsification treatment to prepare the o/w emulsion. The emulsification treatment may be carried out using any suitable means known in the art such as mechanical stirring, high speed shearing, ultrasonic emulsifying, high pressure homogenizing or microfluidizer, preferably mechanical stirring, high speed shearing. It has been found advantageous to control the stirring speed during the homogenization step to adjust the microparticle size. In general, smaller microparticle sizes are achieved by applying a higher homogenization speed (v_{H}). Typically, the homogenization speed (v_{H}) for the emulsification is in the range of from 1000 to 20000 rpm. In an embodiment of the process according to the invention a homogenization speed of from 6000 to 15000 rpm, preferably of from 8000 to 15000 rpm, particularly preferably of from 11000 to 15000 rpm, is applied. The emulsification treatment is preferably carried out under such conditions as to produce an O/W emulsion in which most of the oil droplets contained therein have an average diameter of about 0.5 to 50 μm. Typically, the total time for the homogenization step is in the range of 20-60 seconds. This time frame includes the injection of phase (a) into phase (b), which typically is being performed within 1-20 seconds, preferably within 5-15 seconds, particularly preferably within 10 seconds. In an embodiment of the process according to the invention the total homogenization time is 20-30 seconds, preferably 30 seconds of which during the first 10 seconds phase (a) is injected into phase (b).

In the emulsification treatment, one or more additives selected from surfactants and water-soluble polymers which contribute to the stabilization of the O/W emulsion may be added to the mixed system. Usable surfactants include those from the group of anionic emulsifiers (e.g. sodium lauryl sulfate, sodium stearate), cationic emulsifiers (e.g. cetyl pyridinium chloride), amphoteric emulsifiers (e.g. lecithin), and non-ionogenic emulsifiers (e.g. macrogol stearate, macrogol sorbitan oleate, polyvinylalcohol polyvinyl pyrrolidone, carboxymethylcellulose, hydroxypropylcellulose, gelatin and the like). These additives may be used at such a concentration as to give a 0.01 to 10% (w/w) aqueous solution.

Steps (ii) and (iii):

The emulsion resulting from the emulsification treatment of step (i) may be stirred for a further time period, typically to support the microparticle formation or to achieve a higher efficiency for the extraction of the porogenic agent. When volatile solvents are employed, such an optional stirring step may also be combined with step (iii) of the inventive process and thereby serve to remove the volatile solvents via evaporation. Solvent evaporation may further be supported by application of a reduced pressure. The time period for the stirring and stirring speed may then favorably be chosen to achieve the desired degree of evaporation of the solvent. In a particular embodiment of the inventive process the emulsion resulting from step (i) is typically stirred for a time period of 1 to 10 hours, preferably of 2 to 7 hours while applying a stirring speed (vs) of typically 300 to 2000 rpm, preferably 500 to 1000 rpm. In a typical procedure the emulsion is stirred for about 5 h while applying a speed of about 800 rpm.

### Step (iv):

The porous microparticles are typically separated from the remaining phase via centrifugation, filtration or sedimentation. In a particular embodiment of the inventive process the microparticles are separated by centrifugation, typically with a centrifugation speed of 1000 rpm to 5000 rpm, for a time period of 5 to 15 minutes. In a typical procedure a centrifugation speed of 4000 rpm for a time period of 10 minutes is applied.

The resulting microparticle pellet may then be typically washed with distilled water, e.g. to remove the residues of e.g. emulsifier adsorbed on microspheres surface during the preparation process. Typically this may be performed by addition of water, followed by mixing and centrifugation (1000 rpm to 5000 rpm, preferably of 4000 rpm for a time period of 5-15 minutes, preferably of 10 minutes, and thereafter decanting of water).

### Step (v):

In a particular embodiment of the inventive process the collected microparticles are subjected to lyophilisation in order to completely dry the microparticles. Drying the particles may be particularly advantageous to reduce aggregation and improve flowability and good dispersibility of the resulting microparticle powder.

### Porous Microparticles

The term “porous microparticle” is used herein to refer generally to a variety of structures having sizes from about 10 nm to 2000 μm (2 mm) and includes microcapsules, microspheres, nanoparticles, nanocapsules, nanospheres as well as in general particles, that are less than about 2000 μm (2 mm). The microparticles may or may not be spherical in shape. In a preferred embodiment the porous microparticles are spherical in shape. Furthermore, the term “porous microparticles” refers to particles that are interspersed with pores of various sizes and numbers. In a preferred embodiment of the invention the pores pervade the entire volume of the microparticles.

The aerodynamic behavior as well as the drug release rate of the porous microparticles and the pharmaceutical preparations according to the invention may be controlled and adjusted by controlling microparticle composition and thereby microparticle geometric size, and/or microparticle porosity. For a given composition and particle size, the porosity and the release rate of the microparticles is in turn dependent on the ratio and type of the polyvinylpyrrolidone and/or polyvinylpyrrolidone derivative employed as a porogenic agent within the inventive process.

Porosity (ε) is the ratio of the volume of voids contained in the microparticles (Vᵥ) to the total volume of the microparticles (Vₜ):

ε=Vᵥ/Vₜ (EQ. 2)

This relationship can be expressed in terms of the envelope density (ρₑ) of the microparticles and the absolute density (ρₐ) of the microparticles:

ε=1−ρₑ/ρₐ (EQ. 3)

The absolute density is a measurement of the density of the solid material present in the microparticles, and is equal to the mass of the microparticles (which is assumed to equal the mass of solid material, as the mass of voids is assumed to be negligible) divided by the volume of the solid material (i.e., excludes the volume of voids contained in the microparticles and the volume between the microparticles). Absolute density can be measured using techniques such as helium pycnometry. The envelope density is equal to the mass of the microparticles divided by the volume occupied by the microparticles (i.e., equals the sum of the volume of the solid material and the volume of voids contained in the microparticles and excludes the volume between the microparticles). Envelope density can be measured using techniques such as mercury porosimetry or using a GeoPyc™ instrument (Micromeritics, Norcross, Ga.). However, such methods are limited to geometric particle sizes larger than desirable for pulmonary applications. The envelope density can be estimated from the tap density (pt) of the microparticles. The tap density is a measurement of the packing density and is equal to the mass of microparticles divided by the sum of the volume of solid material in the microparticles, the volume of voids within the microparticles, and the volume between the packed microparticles of the material. Tap density can be measured using a GeoPyc™ instrument or techniques such as those described in the British Pharmacopoeia and ASTM standard test methods for tap density. It is known in the art that the envelope density can be estimated from the tap density for essentially spherical microparticles by accounting for the volume between the microparticles:

ρₑ=ρₜ/0.794 (EQ. 4)

The porosity can be expressed as follows:

ε=1−ρₜ/(0.794*ρₐ) (EQ. 5)

As used herein, the terms “size”, “diameter” or “D” in reference to particles refers to the number average particle size, unless otherwise specified. An example of an equation that can be used to describe the number average particle size is shown below:

$$D = \frac{{\sum }_{i = 1}^{p} {n}_{i} {D}_{i}}{{\sum }_{i = 1}^{p} {n}_{i}} \qquad ( EQ . 6 )$$

where nᵢ=number of particles of a given diameter (Dᵢ).

As used herein, the terms “geometric size,” “geometric diameter,” “volume average size,” “volume average diameter”, “volume mean diameter” or “D_{g}” refers to the volume weighted diameter average.

An example of an equation that can be used to describe the volume mean diameter (D[4,3]), which is most commonly used for laser diffraction particle analysis, is shown below:

$$D [ 4 , 3 ] = \frac{{\sum }_{1}^{n} {D}_{i}^{4} {v}_{i}}{{\sum }_{1}^{n} {D}_{i}^{3} {v}_{i}} \qquad ( EQ . 7 )$$

where Dᵢ represents diameter, and vᵢ represents the relative amount of particles with diameter Dᵢ, in relation to all particles.

As used herein, the term “volume median” refers to the median diameter value of the volume-weighted distribution. The median is the diameter for which 50% of the total are smaller and 50% are larger, and corresponds to a cumulative fraction of 50%.

Geometric particle size analysis can be performed on a Coulter counter, by light scattering, by light microscopy, scanning electron microscopy, or transmission electron microscopy, laser diffraction methods, or time-of-flight methods, as known in the art.

As used herein, the term “aerodynamic diameter” refers to the equivalent diameter of a sphere with density of 1 g/mL, were it to fall under gravity with the same velocity as the particle analyzed. The aerodynamic diameter (Dₐ, MMAD) of a microparticle is related to the geometric diameter (D_{g}) and the envelope density (ρₑ) by the following:

Dₐ=D_{g}\sqrt{ρₑ} (EQ. 8)

Porosity affects envelope density (EQ. 8) which in turn affects aerodynamic diameter. Thus porosity can be used to affect both where the microparticles go in the lung and the rate at which the microparticles release the pharmaceutically active agent in the lung. Gravitational settling (sedimentation), inertial impaction, Brownian diffusion, interception and electrostatic precipitation affect particle deposition in the lungs. Gravitational settling and inertial impaction are dependent on da and are the most important factors for deposition of particles with aerodynamic diameters between 1 μm and 10 μm. Particles with Dₐ>10 μm will not penetrate the tracheobronchial tree, particles with Dₐ in the 3-10 μm range have predominantly tracheobronchial deposition, particles with Dₐ in the 1-3 μm range are deposited in the alveolar region (deep lung), and particles with Dₐ<1 μm are mostly exhaled. Respiratory patterns during inhalation can shift these aerodynamic particle size ranges slightly. For example, with rapid inhalation, the tracheobronchial region shifts to between 3 μm and 6 μm. It is a generally held belief that the ideal scenario for delivery to the lung is to have Dₐ<5-6 μm. Aerodynamic diameters can be determined on the dry powder using an Aerosizer (TSI), which is a time of flight technique, or by cascade impaction, or liquid impinger techniques. As used herein, the terms “Fine Particle Fraction” or “respirable dose” refer to a dose of drug that has an aerodynamic size such that particles or droplets comprising the drug are in the aerodynamic size range that would be expected to reach the lung upon inhalation. Fine particle fraction/respirable dose can be measured using a next generation cascade impactor (NGI), a liquid impinge, or time of flight methods (as described by United States Pharmacopeia [USP34_NF29 Chapter <601>Aerosols, Nasal Sprays, Metered-Dose Inhalers, and Dry Powder Inhalers Monograph, The United States Pharmacopoeia and The National Formulary: The Official Compendia of Standards. The United States Pharmacopeial Convention, Inc., Rockville, Md., USA. 2011. Apparatus 6] and European Pharmacopoeia [Ph. Eur. Section 2.9.18, Preparations for inhalation: Aerodynamic assessment of fine particles, European Pharmacopoeia: 7th Ed., Council of Europe, 67075, Strasbourg, France. 2010].

For pulmonary administration, the porous microparticles preferably have an experimentally determined aerodynamic diameter (MMAD) of between 1 μm and 10 μm. The porous microparticles obtainable via the process according to the invention typically have an MMAD of from 1 to 8 μm. In one embodiment according to the invention, the porous microparticles have a volume average diameter (D[4,3]) from about 7 μm to 25 μm. In another embodiment, the porous microparticles have a volume average diameter (D[4,3]) from about 9 μm to 15 μm.

In a further aspect the porous microparticles comprise the pharmaceutically active agent encapsulated, microencapsulated, or otherwise contained within the microparticles. For example, the microparticles can comprise 0.01% to about 90% API relative to the sum of amounts of matrix and API, weight to weight (w/w), including without limitation, about 0.1%, 0.5%, 1%, 5%, 10%, 15%, 20%, 30%, 40%, 50%, 60%, 70%, or 80% relative to the sum of amounts of matrix and API (w/w), including any range between the disclosed percentages. In a preferred embodiment according to the invention the pharmaceutically active agent is used in an amount of 1 to 10% relative to the sum of amounts of matrix and API (w/w).

### Sustained Release

The term “sustained release” indicates that, for example, after 12 hours, less than 60% of the active agent or active agent fraction has been released. Alternatively, it may indicate that, after 24 hours, less than 70% of the active agent or active agent fraction has been released. In a preferred embodiment according to the invention the porous microparticles release the pharmaceutically active agent in a sustained manner. Under experimental conditions described in the methods section, preferably, less than 60% of encapsulated API is released within 12 hours, and less than 70% of API is released within 24 hours after start of the dissolution experiment.

In a preferred embodiment according to the invention, a therapeutically effective amount of the pharmaceutically active agent is released from the porous microparticles in the lungs for at least 2 hours, preferably for at least 12 hours, most preferably for at least 24 hours, for at least particularly preferable 168 hours.

For a given microparticle composition (pharmaceutically active agent and matrix material) and structure (microparticle porosity and thus density) an iterative process can be used to define where the microparticles go in the lung and the duration over which the microparticles release the pharmaceutically active agent: 1) the matrix material, the pharmaceutically active agent content, and the microparticle geometric size are selected to determine the time and amount of initial pharmaceutically active agent release; 2) the porosity of the microparticles is selected to adjust the amount of initial pharmaceutically active agent release, and to ensure that significant release of the pharmaceutically active agent occurs beyond the initial release and that the majority of the pharmaceutically active agent release occurs within a given time period; and then 3) the geometric particle size and the porosity are adjusted to achieve a certain aerodynamic diameter which enables the particles to be deposited by inhalation to the region of interest in the lung.

As used herein, the term “initial release” refers to the amount of pharmaceutically active agent released shortly after the microparticles become wetted. The initial release upon wetting of the microparticles may result from pharmaceutically active agent which is not fully encapsulated and/or pharmaceutically active agent which is located close to the exterior surface of the microparticle. The amount of pharmaceutically active agent released in the first thirty minutes is used as a measure of the initial release.

### Pharmaceutical Composition

The pharmaceutical composition according to the invention can be administered as the sole pharmaceutical composition or in combination with one or more other pharmaceutical compositions or active agents where the combination causes no unacceptable adverse effects.

“Combination” means for the purposes of the invention not only a dosage form which contains all the active agents (so-called fixed combinations), and combination packs containing the active agents separate from one another, but also active agents which are administered simultaneously or sequentially, as long as they are employed for the prophylaxis or treatment of the same disease.

“Fixed dose combination” as used herein refers to a pharmaceutical product that contains two or more active ingredients that are formulated together in a single dosage form available in certain fixed doses.

Besides the aforementioned polymer materials and surfactants, it may be desirable to add other excipients to a particulate composition to improve particle rigidity, production yield, emitted dose and deposition, shelf-life and patient acceptance. Such optional excipients include, but are not limited to: coloring agents, taste masking agents, salts, hygroscopic agents, antioxidants, and chemical stabilizers. Further, various excipients may be incorporated in, or added to, the particulate matrix to provide structure and form to the particulate compositions.

The terms “pulmonary”, “pulmonary delivery” or “pulmonary drug delivery” refer to all manners of delivery wherein a drug substance, which is preferably encapsulated in the porous microparticles according to the invention, is brought into direct contact with all or any portion of the respiratory tract, including the lower respiratory tract. In the sense of the present invention the porous microparticles can be formulated so as to be suitable for aerosolization or for dry powder inhalation, preferably for dry powder inhalation. The formulated porous microparticle size can be varied according to the size deemed to be optimal for pulmonary delivery.

Suitable inhalers comprise dry powder inhalers (DPIs). Same such inhalers include unit dose inhalers, where the dry powder is stored in a capsule or blister, and the patient loads one or more of the capsules or blisters into the device prior to use. Other multi-dose dry powder inhalers include those where the dose is pre-packaged in foil-foil blisters, for example in a cartridge, strip or wheel. Other multi-dose dry powder inhalers include those where the bulk powder is packaged in a reservoir in the device.

As used herein, the term “emitted dose” or “ED” refers to an indication of the delivery of dry powder from a suitable inhaler device after a firing or dispersion event from a powder unit or reservoir. ED is defined as the ratio of the dose delivered by an inhaler device (described in detail below) to the nominal dose (i.e., the mass of powder per unit dose placed into a suitable inhaler device prior to firing). The ED is an experimentally-determined amount, and is typically determined using an in vitro device set up which mimics patient dosing. To determine an ED value, a nominal dose of dry powder (as defined above) is placed into a suitable dry powder inhaler, which is then actuated, dispersing the powder. The resulting aerosol cloud is then drawn by vacuum from the device, where it is captured on a tared filter attached to the device mouthpiece. The amount of powder that reaches the filter constitutes the delivered dose. For example, for a 5 mg, dry powder-containing blister pack placed into an inhalation device, if dispersion of the powder results in the recovery of 4 mg of powder on a tared filter as described above, then the ED for the dry powder composition is: 4 mg (delivered dose)/5 mg (nominal dose)×100=80%.

For administration to the pulmonary system using a dry powder inhaler, the porous microparticles according to the invention can be combined (e.g. blended) with one or more pharmaceutically acceptable bulking agents and administered as a dry powder blend formulation. The bulking agent can, for example, comprise particles which have a volume average size between 10 and 500 μm. Examples of pharmaceutically acceptable bulking agents include sugars such as mannitol, sucrose, lactose, fructose and trehalose and amino acids Amino acids that can be used include glycine, arginine, histidine, threonine, asparagine, aspartic acid, serine, glutamate, proline, cysteine, methionine, valine, leucine, isoleucine, tryptophan, phenylalanine, tyrosine, lysine, alanine, and glutamine. In a preferred embodiment according to the invention the bulking agents are selected from the group consisting of lactose, mannitol, sorbitol, trehalose, xylitol, and combinations thereof.

### Use in Therapy

The present invention also relates to a use of the pharmaceutical composition for pulmonary drug delivery, preferably via inhalation, to treat or prevent disorders, preferably pulmonary diseases or conditions of the lungs and/or airways, wherein the pharmaceutical composition comprises the porous microparticles comprising a pharmaceutically effective amount of a pharmaceutically active agent according to the invention. The present invention also relates to a method for treating or preventing a preferably pulmonary disease or condition of the lungs and/or airways, comprising pulmonary administration of the pharmaceutical composition, preferably via inhalation, wherein the composition comprises the porous microparticles comprising a pharmaceutically effective amount of an active agent according to the present invention.

Examples of pulmonary diseases or conditions of the lungs and/or airways according to the invention include but are not limited to chronic pulmonary diseases, lung cancer, cystic fibrosis, idiopathic pulmonary fibrosis, asthma, bronchitis, pneumonia, pleurisy, emphysema, pulmonary fibrosis, diabetes, interstitial lung disease, sarcoidosis, chronic obstructive pulmonary disease (COPD), asthma, infant respiratory distress syndrome, adult respiratory distress syndrome, pulmonary actinomycosis, pulmonary alveolar proteinosis, pulmonary anthrax, pulmonary arteriovenous malformation, pulmonary edema, pulmonary embolus, pulmonary histiocytosis X (eosinophilic granuloma), pulmonary nocardiosis, pulmonary tuberculosis, pulmonary venoocclusive disease, rheumatoid lung disease, and others.

Preference is given in particular to pulmonary hypertension, chronic obstructive pulmonary disease (COPD), pulmonary fibrosis and lung cancer.

The present invention also relates to the use of any pharmaceutical composition described herein in the manufacture of a medicament for the treatment of treating diseases or conditions of a patient or subject, such as diseases or conditions of the lungs and/or airways.

The present invention also provides any dry powder formulation herein comprising respirable porous microparticles for use in the treatment of diseases or conditions of a patient or subject, such as diseases or conditions of the lungs and/or airways.

The present invention also relates to a use of the pharmaceutical composition according to the invention to treat or prevent disorders, preferably pulmonary hypertension, chronic obstructive pulmonary disease (COPD), pulmonary fibrosis and lung cancer.

In the context of the present invention, the term “pulmonary hypertension” encompasses both primary and secondary subforms thereof, as defined below by the Dana Point classification according to their respective aetiology [see D. Montana and G. Simonneau, in: A. J. Peacock et al. (Eds.), Pulmonary Circulation. Diseases and their treatment, 3^{rd} edition, Hodder Arnold Publ., 2011, pp. 197-206; M. M. Hoeper et al., J. Am. Coll. Cardiol. 2009, 54 (1), S85-S96]. These include in particular in group 1 pulmonary arterial hypertension (PAH), which, among others, embraces the idiopathic and the familial forms (IPAH and FPAH, respectively). Furthermore, PAH also embraces persistent pulmonary hypertension of the newborn and the associated pulmonary arterial hypertension (APAH) associated with collagenoses, congenital systemic pulmonary shunt lesions, portal hypertension, HIV infections, the intake of certain drugs and medicaments (for example of appetite supressants), with disorders having a significant venous/capillary component such as pulmonary venoocclusive disorder and pulmonary capillary haemangiomatosis, or with other disorders such as disorders of the thyroid, glycogen storage diseases, Gaucher disease, hereditary teleangiectasia, haemoglobinopathies, myeloproliferative disorders and splenectomy. Group 2 of the Dana Point classification comprises PH patients having a causative left heart disorder, such as ventricular, atrial or valvular disorders. Group 3 comprises forms of pulmonary hypertension associated with a lung disorder, for example with chronic obstructive lung disease (COPD), interstitial lung disease (ILD), pulmonary fibrosis (IPF), and/or hypoxaemia (e.g. sleep apnoe syndrome, alveolar hypoventilation, chronic high-altitude sickness, hereditary deformities). Group 4 includes PH patients having chronic thrombotic and/or embolic disorders, for example in the case of thromboembolic obstruction of proximal and distal pulmonary arteries (CTEPH) or non-thrombotic embolisms (e.g. as a result of tumour disorders, parasites, foreign bodies). Less common forms of pulmonary hypertension, such as in patients suffering from sarcoidosis, histiocytosis X or lymphangiomatosis, are summarized in group 5.

It should be apparent to one of ordinary skill in the art that changes and modifications can be made to this invention without departing from the spirit or scope of the invention as it is set forth herein.

All publications, applications and patents cited above and below are incorporated herein by reference.

The weight data are, unless stated otherwise, percentages by weight and parts are parts by weight.

### FIGURES

FIG. 1. In vitro release profiles of Cinaciguat loaded large porous particles and non-porous particles.

FIG. 2. In vitro release profiles of Budesonide loaded LPPs prepared with different amount/type of polyvinylpyrrolidone.

FIG. 3. In vitro aerodynamic diameter distribution of LPPs and the control formulation tested by NGI.

FIG. 4. Surface morphology of Cinaciguat loaded LPPs prepared with PLGA 502 as the matrix and PVP K12 as porogen.

FIG. 5. Surface morphology of Cinaciguat loaded LPPs prepared with PLGA 503H as the matrix and PVP K12 as porogen.

FIG. 6. Surface and internal morphology of Cinaciguat loaded LPPs prepared with PLGA 503 as the matrix and PVP K12 as porogen.

FIG. 7. Surface morphology of Cinaciguat loaded LPPs prepared with PLGA 502 as the matrix and F-127 as porogen.

FIG. 8. Surface morphology of Budesonide loaded LPPs prepared with PLGA 503 as the matrix and PEG4000 as porogen.

FIG. 9. Influence of Cinaciguat loaded LPPs and the control formulation on pulmonal arterial blood pressure (PAP) in a mini-pig model.
Influence of Cinaciguat loaded LPPs and the control formulations on systemic blood pressure (BP) in a mini-pig model.

FIG. 10. Influence of Cinaciguat loaded LPPs and the control formulation on pulmonal arterial blood pressure (mPAP) in a mini-pig model.
Influence of Cinaciguat loaded LPPs and the control formulations on systemic blood pressure (mBP) in a mini-pig model.

Additional embodiments and features are set forth in part in the description that follows, and in part will become apparent to those skilled in the art upon examination of the specification or may be learned by the practice of the invention. This invention is further illustrated by the following examples which should not be construed as limiting.

### EXAMPLES

### Abbreviations Used in the Examples

ABC ammonium bicarbonate
ACN acetonitrile
API active pharmaceutical ingredient; pharmaceutically active agent
DCM dichloromethane; methylene chloride
HPLC high performance liquid chromatography
HPMC hydroxypropyl methylcellulose
MOC micro-orifice collector
NMP 1-N-methyl-2-pyrrolidone
NGI NEXT GENERATION IMPACTOR™ (cascade impactor)
PBS phosphate buffered saline
Ph. Eur. European Pharmacopoeia
PVA polyvinyl alcohol
PVP polyvinylpyrrolidone
SD standard deviation
SDS sodium dodecyl sulphate
SEM scanning electron microscopy
S/N signal-to-noise ratio
USP United States Pharmacopoeia
X-RPD X-ray powder diffraction

### Statistical Analysis

All the experimental results were depicted as mean value±standard deviation (SD) from at least three measurements (unless otherwise specified). Significance of difference was evaluated using one-way analysis of variance (ANOVA) at a probability level of 0.05.

### Raw Materials:

Acetonitrile: (HPLC grade; Shandong Yuwang Co., Ltd, Shandong, China)
Ammonium dihydrogen phosphate: (A.R.; Tianjin Bodi Chemical Corporation, Tianjin, China)
Diammonium hydrogen phosphate: (A.R.; Tianjin Bodi Chemical Corporation, Tianjin, China)
Dichloromethane: (HPLC grade; Tianjin Kemiou Chemical Co., Ltd, Tianjin, China)
Cinaciguat: 4-[((4-carboxybutyl)-{2-[(4-phenethylbenzyl)oxy]phenethyl}amino)methyl]benzoic acid; Bayer Pharma. AG, Wuppertal, Germany
Budesonide: 16α,17-[(R,S)-Butan-1,1-diyldioxy]-11β,21-dihydroxypregna-1,4-dien-3,20-dion, CAS [84371-65-3], (Hubei Gedian Humanwell Pharmaceutical Co., Ltd)
Pluronic® F-127: CAS [9003-11-6], (BASF Co., Ltd in China)
Polyvinyl alcohol: (PVA 205 and 217, partially hydrolyzed, Kuraray Co., Ltd. in Japan)
Polyvinylpyrrolidone: (PVP K12, K17, K30; International Specialty Products)
PLGA: (Resomer® RG 502, 502H, 503H, Evonik, Essen, Germany)
Sodium dodecylsulfate (SDS): (BASF Co., Ltd in China)
TWEEN® 20: Tianjin Bodi Chemical Corporation, Tianjin, China

All other chemicals were of analytical grade.

### Methods

### A) HPLC Methods

### Method 1: HPLC Method for the Determination of the Content of Cinaciguat

10 μl of each sample was injected into an HP 1100 HPLC system (Agilent, Waldbronn, Germany) and samples were run on a heated (40° C.) Gemini C18 column (with pre-column; 150×4:6 mm; particle size: 5 μm; Phenomenex), applying a flow rate of 1 ml/min. Equilibration time was 10.00 min. The mobile phase consisted of a mixture of acetonitrile (A) and ammonium phosphate buffer pH 7.4 (B). The following gradient was applied: 0.0 min 5% A/95% B→9 min 35% A/65% B→25 min 80% A/20% B→35 min 80% A/20% B→35 min 80% A/20% B.

Cinaciguat was detected using a diode array detector (DAD) at a wavelength of 230 nm. The drug retention time of cinaciguat was in the range of 14.7-14.8 min. Cinaciguat content (column 3 in Tables below) was quantified by using an external 2-point calibration straight line. Precision of the system was determined with each sample set run, by six times injection of one cinaciguat standard, coefficient of variation of peak areas resulting from these six injections was always below 2%. Relative Y-axis intercept of a 2-point calibration straight line was always below 3% (referring to all Cinaciguat standards).

Determination of cinaciguat content: Concentration (C) and peak area (A) show a linear correlation in the applied concentration range (0.995-398.0 μg/mL; A=23.832 C+4.6534, R²=0.9998, n=8).

Sensitivity of the method: The limit of quantitation (LOQ, defined as the concentration of the drug giving a S/N=10) was 0.6-1 ng and the limit of detection (defined as S/N=3) was 0.2 ng.

### Method 2: HPLC Method for the Determination of the Content of Budesonide

10 μl of each sample was injected into an HP 1100 HPLC system (Agilent, Waldbronn, Germany) and samples were run on a heated (40° C.) ZORBAX SB-C18 column (with pre-column; 150×4.6 mm; particle size: 5 μm; Agilent), applying an isocratic elution with a flow rate of 1 ml/min and mobile phase consisting of a mixture of water/methanol (28/72, v/v).

Budesonide was detected using a variable wavelength detector (VWD) at a wavelength of 248 nm. The drug retention time of budesonide was in the range of 6.6-6.7 min.

Budesonide content was quantified by using an external 2-point calibration straight line. Precision of the system was determined with each sample set run, by six times injection of one budesonide standard, coefficient of variation of peak areas resulting from these six injections was always below 2%. Relative Y-axis intercept of a 2-point calibration straight line was always below 3% (referring to all budesonide standards).

Determination of budesonide content: Concentration (C) and peak area (A) show a linear correlation in the applied concentration range (1-100.0 μg/mL; A=38.526 C-5.4802, R²=0.9996, n=8).

Sensitivity of the method: The limit of quantitation (LOQ, defined as the concentration of the drug giving a S/N=10) was 1.5 ng and the limit of detection (defined as S/N=3) was 0.5 ng.

### B) Determination of the Drug Loading

Drug loading (% DL) and encapsulation efficiency (% EE) were calculated as:

Drug Loading (% DL)=amount of API (weight)/amount of API+excipient (weight)*100[%]

Encapsulation efficiency (% EE)=measured drug loading/theoretical drug loading*100[%]

### Method 1: Cinaciguat

To a volumetric flask (50.0±0.05 mL) were added 10 mg of cinaciguat loaded microparticles followed by addition of 2.5 ml NMP to dissolve the particles. Then, 47.5 ml of ACN was added and the resulting mixture vortexed until a homogenous mixture was obtained. Cinaciguat quantities were measured by injecting 10 μl of the supernatant onto HPLC as described in the methods.

### Method 2: Budesonide

To a volumetric flask (10.0±0.02 mL) were added 5 mg of budesonide loaded microparticles followed by addition of 0.5 ml DCM to completely dissolve the particles and then methanol to 10.0 mL to precipitate PLGA. The mixture was then centrifugated and the concentration of budesonide in the supernatant was subsequently determined by the HPLC Methods described above. Drug loading (% DL) and encapsulation efficiency (% EE) were determined the same as cinaciguat.

### C) Determination of the Particle Size

Particle size distribution and volume mean diameter D(4,3) of the microparticles were determined using a laser diffraction particle size analyzer (Beckman-Coulter LS 230, USA). About 10 mg of the microparticles were dispersed in 0.1% PVA 205 solution and then measured. Particle size is expressed as volume mean diameter+SD of values collected from three different batches.

D) Determination of Tapped Density (ρₜ) and Theoretical Mass Mean Diameter (MMADₜ)

Theoretical mass mean aerodynamic diameters (MMADₜ) of the particles were calculated from measured tapped density values (ρₜ) of the particles. To determine tapped density (ρₜ), an aliquot of 100 mg microspheres was transferred to a 10 (±0.05) ml graduated cylinder. Tapped density of the particles (ρₜ, [g/cm³]) was calculated as the ratio between sample weight [g] and the volume [ml] occupied after 200-500 times tapping according to the following equation (EQ. 9):

$${ρ}_{t} [ g \text{/} {cm}^{3} ] = \frac{Sample Weight ( g )}{Final Volume after taps ( mL )} \times 100 \% \qquad ( EQ . 9 )$$

The theoretical mass mean aerodynamic diameter (MMADₜ) of the microparticles was calculated according to the following equation (EQ. 10):

$${MMAD}_{t} [ µm ] = D ( 4 , 3 ) \sqrt{\frac{{ρ}_{t}}{{ρ}_{0} χ}} \qquad ( EQ . 10 )$$

Where “ρₜ” is the tapped density, “ρ₀” is the reference density for a sphere (ρ₀=1 g/cm³), and “χ” is a shape factor (for a spherical particle χ=1).

### E) Determination of the In Vitro Release Rates of the Drug Loaded Porous Microparticles

### Method 1: Cinaciguat

Cinaciguat loaded microparticles (10 mg) were dispersed in 5.0 ml of 0.1% (w/v) SDS containing phosphate buffer (10 mM PBS (sodium hydrogen phosphate/sodium dihydrogen phosphate), pH=7.4, 0.02% (w/v) sodium azide). Dissolution experiments were performed under non-sink conditions, meaning in 2-3 fold volume of dissolution medium that would form a saturated solution at given cinaciguat amount per dissolution vial. (cf sink conditions definition by United States Pharmacopoea USP35, General Information/<1092>. The Dissolution Procedure: Development and Validation): dissolution in 3-10 fold volume that would form a saturated API solution). The mixture was continuously agitated (80 rpm) in an incubator/shaker (37±1° C.). At predetermined time intervals (2 h, 4 h, 8 h, 24 h, 48 h, 72 h, 120 h, 168 h) samples were withdrawn and centrifuged (4000 rpm, 10 min). The supernatant was decanted and a sample was analysed by HPLC as described in the methods. The microparticles were then resuspended in fresh medium of equal volume.

### Method 2: Budesonide

10 mg of budesonide loaded microparticles were dispersed in 5.0 ml of 0.15-0.2% (w/v) SDS containing phosphate buffer (10 mM PBS (sodium hydrogen phosphate/sodium dihydrogen phosphate), pH=7.25-7.30, 0.02% (w/v) sodium azide). The mixture was continuously agitated (80 rpm) in an incubator/shaker (37±1° C.). At predetermined time intervals (2 h, 4 h, 8 h, 24 h, 48 h, 72 h, 96 h, 120 h, 144 h) samples were withdrawn and centrifuged (4000 rpm, 10 min). The supernatant was decanted and a sample was analysed by HPLC as described in the methods. The microparticles were then resuspended in fresh medium of equal volume.

F) In vitro aerosolization analysis by Next Generation Impactor (NGI); Determination of MMADₑ, FPF

The in vitro aerosolization analysis was performed in accordance with pharmacopeia guidelines: USP34_NF29, Chapter <601>; Ph. Eur. Chapter 2.9.18.

The in vitro aerosolization performance of the microparticles from a dry powder inhaler (DPI, Cyclohaler, PH&T, Milano, Italy) using VCAPS Plus HPMC capsules (Capsugel, Greenwood, USA) was characterized using a NEXT GENERATION IMPACTOR™ (NGI, MSP Corporation, USA) with a stainless steel induction port (i.e. USP throat) and pre-separator attachments. The impactor was equipped with a critical flow controller (Copley TPK 2000), a flow meter (Copley DFM 2000) and a vacuum pump (Copley HCPS, all Copley Scientific, UK).

Prior to the measurement, to decrease particle re-entrainment, the NGI impactor plates were coated with a thin film of ethanolic 10% TWEEN® 20 (w/v) solution and left in a fuming hood for 30 min to evaporate the ethanol. HPMC capsules (size 3, Capsugel, Greenwood, USA) were filled with 20 mg of microparticles and placed in a Cyclohaler® (Pharbita BV, the Netherlands) tightly connected to the NGI equipment. For each experiment, ten individual capsules were discharged into the NGI at a final flow rate of 100 L/min (by adjusting ΔP₁ and P₂/P₃ (ΔP₁=4 kPa, P₂/P₃<0.5, as described in United States Pharmacopoe General Chapters 601) and an actuation time of 2.4 s/per capsule. The cut-off particle aerodynamic diameters of each stage of the impactor were: pre-separator (10.0 μm), stage 1 (6.12 μm), stage 2 (3.42 μm), stage 3 (2.18 μm), stage 4 (1.31 μm), stage 5 (0.72 μm), stage 6 (0.40 μm), and stage 7 (0.24 μm). Furthermore, the NGI was equipped with a micro orifice collector (MOC; D₈₀, 0.07 μm), which acts as a final filter. The MOC used was configured to collect 80% of particles of >70 nm in size.

After actuation, all the individal components (device, capsules, throat, pre-separator and all impactor plates containing microparticles) were washed with NMP and the solvent fractions were collected into separate volumetric flasks (50.0±0.05 mL), diluted to the final volume with ACN and analyzed by HPLC as described in the methods. The experimental mass median aerodynamic diameter (MMADₑ) and the geometric standard deviation (GSD) were obtained by a linear fit of the cumulative percent less than the particle size range by weight plotted on a probability scale as a function of the logarithm of the effective cut-off diameter (see USP general chapter 601).

The fine particle fraction (FPF) is considered the fraction of particles that is available for lung deposition. Thus, FPF is the ratio of mass of particles in the aerosol with aerodynamic diameter (Dₐ) less than equal to ˜5 μm, to the total mass of emitted particles. In this context, FPF was determined by calculating the mass of particles in stages 1 to 5 (0.72 μm to 6.12 μm) from the sum of API amounts in stages 1-5 and drug loading, and dividing the mass of particles in stages 1 to 5 by the total mass of emitted particles (EQ. 11).

GSD values were calculated according to below equation (EQ. 12). These values can be similarly obtained from the bundled software of the Next Generation Cascade Impactor (NGI, Copley Scientific Inc.).

$$\begin{matrix}FPF \% = \frac{{M}_{stage 1 through 5}}{{M}_{total} ( Induction port + Pre \text{-} separator + all stages )} & ( EQ . 11 ) \\ GSD = \sqrt{\frac{{D}_{84.14 \%}}{{D}_{15.87 \%}}} & ( EQ . 12 )\end{matrix}$$

where

M_{stage 1 through 5} is the sum of mass of API (thus the sum of mass of aerolized particles, assuming content uniformity) found in each of the NGI collector stages 1, 2, 3, 4, and 5.

Mₜₒₜₐₗ is the sum of mass of API (thus the sum of aerolized particles, assuming content uniformity) found in all parts of the NGI (induction port, pre-separator, all collector stages).

### G) Differential Scanning Calorimetry (DSC)

Thermodynamic analysis of the microparticle formulations was performed with differential scanning calorimetry (DSC-1, Mettler-Toledo, Gießen, Germany). Samples of the microparticles (3-5 mg) were placed in hermetically sealed aluminum pans. The samples were scanned at a heating rate of 10° C./min from −20° C. to 200° C. under nitrogen atmosphere. Respective thermic events (e.g. glass transition points of polymer, and/or melting points of APIs) were recorded, respectively.

### H) X-Ray Powder Diffraction (X-RPD)

The crystallinity of the microparticles was analyzed using X-ray powder diffraction (XRPD). XRPD patterns were measured using an X-ray diffractometer (X'pert PRO, PANalytical B.V., the Netherlands) with Cu—K_{α} radiation generated at 40 mA and 35 kV. Samples were scanned in a 2θ range of 4.5°-40° with a step size of 0.033° and a counting time of 0.6 s per step.

### I) Scanning Electron Microscopy (SEM)

Morphological examination of microparticles was performed using a scanning electron microscope (SEM) (Hitachi S-3400N, Hitachi Ltd., Japan or Quanta 600, FEI, Hillsboro, USA). A few milligrams (<5 mg) of microparticles was sprinkled onto double-sided adhesive tape attached to an aluminum stub and was then sputter-coated with a thin layer of gold under vacuum. Photographs were taken at varied magnifications (see respective FIGS. 4-8) with an accelerating voltage of 4-5 kV to investigate surface characteristics and morphologies of the microparticles.

### J) Pharmacodynamic Study in Mini-Pig

Minipigs (2-6 kg BW, Göttinger Minipigs® Ellegaard (Ellegaard, Denmark)) were used Animals were sedated by i.m. bolus injection of 25 mg/kg ketamin (Ketavet®, Pfizer Pharmacia GmbH, Berlin, Germany) combined with i.m. bolus injection of 10 mg/kg Azaperon (Stresnil®, Janssen Pharmaceutica, Beerse, Belgien). Anaesthesia was initiated by bolus injection of 1.875 mg/kg ketamin combined with 0.281 mg/kg midazolam (Dormicum®, Roche Pharma AG, Grenzach-Wyhlen, Germany). Anaesthesia was continued and maintained throughout the experiment by infusion of 7.5-30 mg/kg/h ketamin in combination with 1.125-4.5 mg/kg/h midazolam (infusion rate 1-4 ml/kg/h) and 0.2 mg/kg/h Pancuroniumbromid (Pancuronium®, Organon, Oss, Netherland) at an infusion rate of 1 ml/kg/h. Animals were intubated and ventilated (10-12 ml/kg, 35 breath/min; Avea®, Viasys Healthcare, USA, or Engstróm Carestation, GE Healthcare, Freiburg, Germany) to keep an endtidal CO₂-concentration of about 5%. Ventilation is performed with room air enriched with 40% oxygen. For hemodynamic assessment (e.g. pulmonary artery pressure (PAP), blood pressure (BP) and heart rate (HR), catheters were placed in the A. carotis (BP) and a Swan-Ganz®-Catheter is placed via the V. jugularis into the pulmonary artery. Hemodynamics was recorded via pressure transducers (Combitransducer, B. Braun, Melsungen, Germany) and analyzed using Ponemah® aquisition software.

To model pulmonary hypertension after instrumentation a continous infusion of a thromboxan A₂-analogon was started. 0.3-0.75 μg/kg/min 9,11-Dideoxy-9α,11α-epoxymethanoprostaglandin F_{2α} (U-44069; Sigma, Kat.-Nr. D0400, or Cayman Chemical Company, Kat.-Nr. 16440), were solved in physiological NaCl and infused to reach a mean PAP of at least 25 mmHg. The experiment was started when the steady state was reached (usually 30 minutes after the start of the infusion).

The respective powder formulations were then insufflated into the lungs of the minipigs using a specially customized dry powder insufflator device (Dry Powder Insufflator™, DP-4M) and an air pump (AP-1) assembly (Penn-Century, Philadelphia, Pa.) which was advanced via the tubus of the animals.

a) In one experiment the applieddose was 18.75 μg Cinaciguat/kg, which was administered as an LPP microparticle formulation with a drug load of 3.7% (m/m), resulting in an applied formulation dose of 500 μg formulation/kg. For comparison, a powder blend consisting of 5% Cinaciguat (micronized), 20% Lactohale LH 300, and 70% Lactohale LH 200 was administered at a dose of 18.25μ Cinaciguat/kg, corresponding to an applied formulation dose of 375 μg formulation/kg.
b) In a second set of experiments, a three times higher dose of 56.25 μg Cinaciguat/kg was applied, again as an LPP microparticle formulation. In these experiments, LPP formulations with a drug loading of either 3.5% (m/m) and 4.0% (m/m) were used, resulting in applied formulation doses of 1.725 mg/kg and 1.5 mg/kg, respectively. For comparison, a powder blend consisting of 5% Cinaciguat (micronized), 20% Lactohale LH 300, and 70% Lactohale LH 200 was administered at a dose of 56.25 μg Cinaciguat/kg, corresponding to an applied formulation dose of 1.125 mg/kg.

The systemic arterial blood pressure and the pulmonary arterial blood pressure were monitored and recorded as evaluation parameters. The pharmacodynamic evaluation of pulmonary arterial hypertension was investigated in a minipig animal model. The minipigs were anesthetized and the respective powder formulations were insufflated into the lungs of the minipigs using a specially customized dry powder insufflator device (Dry Powder Insufflator™, DP-4M) and an air pump (AP-1) assembly (Penn-Century, Philadelphia, Pa.). A laryngoscope (PennCentury, Philadelphia, Pa.) was used to visualize trachea and epiglottis to ensure a quicker and safer intubation and insertion of the Dry Powder Insufflator™.

At the end of the in-life part of the study, the animals were sacrificed and necropsy was performed. The lungs were removed from the thorax and one lobe was instilled with formalin via the bronchus. Samples of the other lobes were immersion-fixed in formalin. Additionally, lung associated lymph nodes, trachea, heart, liver, thymus, spleen, kidneys, pancreas and adrenals were immersion-fixed in formalin.

The organs were trimmed, embedded in paraffin, cut at 4 μm and stained with hematoxylin and eosin (H&E).

### Formulation Examples

### I. Reference Experiments Comparing Known Porogens/Emulsification Methods

This example illustrates the comparison of known methods of the single/double emulsion-solvent evaporation based preparation of porous microparticles using either a gas forming porogen or an extractable porogen.

I-1. Reference Experiment (1-R): Double-Emulsion Method (w/o/w), Gas-Forming Porogen.

15 mg of cinaciguat were dissolved in 3.0 mL of a mixture of NMP/DCM (1:19, v/v) containing 300 mg PLGA 502. An aqueous solution of ammonium bicarbonate (ABC) (3%, w/v) in 400 μL of water was then dripped into the organic phase under high speed homogenization conditions (T 25 ULTRA-TURRAX®, IKA, Staufen, Germany, 20000 rpm, 30 s) to generate the primary emulsion (w₁/o). The primary emulsion was subsequently injected into 25 mL of a 2% (w/v) aqueous PVA solution (PVA205, partially hydrolyzed) and homogenized at 8000-10000 rpm for 60 s to form the double emulsion (w₁/o/w₂). The double emulsion was then given into 150 mL of 0.2% (w/v) aqueous PVA 205 solution and stirred at room temperature for 5 h to evaporate the volatiles and achieve particle hardening. The particles were collected by centrifugation (4000 rpm, 10 min) and washed three times with distilled water, then lyophilized for 24 h (0.01 atm, −50° C.).

I-2. Reference Experiment (2-R): Single-Emulsion Method (o/w), Extractable Porogen

15 mg of cinaciguat were dissolved in 3 mL of a mixture of NMP/DCM (1:19, v/v) containing 300 mg PLGA 502 and 30.0 mg Pluronic® F-127. The resulting organic phase was then injected into 200 mL of an 0.1% (w/v) aqueous PVA 205 solution. A high-speed homogenizer (T 25 ULTRA-TURRAX®, IKA, Germany) was used for the emulsification operated at a homogenization speed (v_{H}) of 8000-10000 rpm for 30 s. The final emulsion was subsequently stirred at 800 rpm for 5 h at room temperature to evaporate the volatiles. The particles were collected by centrifugation (4000 rpm, 10 min) and washed three times with distilled water, then lyophilized for 24 h (0.01 atm, −50° C.).

The particle parameters were determined as described in the methods section, the test results are collated in Table 3.

TABLE 3

| | PLGA/ | | | | | |
|---|---|---|---|---|---|---|
| | DCM | Porogen/PLGA | PVA | MMADₜ | % EE^{#} | |
| Ex. | (%, w/v) | (w/w) | (%, w/v) | (μm)^{#} | (%) | Porosity^{##} |
| 1-R | 15 | 3.0% (w/v) ABC | 0.1 | 5.2 ± 0.7 | 12.90 ± 0.24 | good |
| 2-R | 15 | 10.0% (w/w) F-127 | 0.1 | 7-8 | 65.72 ± 1.46 | very low |

R: Reference example;
^{#}Given values were determined as described in the methods section.
^{##}Qualitatively assessed from SEM pictures.

Based on literature reports, double emulsion-solvent evaporation methods are commonly used to prepare porous particles. Among the different types of pore forming agents, the use of gas-forming agents is attractive due to their good safety profile, a relatively small amount required and the fine, regular porous structure of the produced microspheres. Porous microparticles were prepared using ammonium bicarbonate as effervescent porogen, where the homogenization conditions were optimised to obtain microparticles with an appropriate MMADₜ value [see e.g. Kim I., Byeon H. J., Kim T. H., Lee E. S., Oh K. T., Shin B. S., Lee K. C., Youn Y. S, Biomaterials, 33, 5574-5583 (2012)]. All the microparticles thus obtained were spherical with homogenous pore distribution over the matrix of the microspheres (qualitative assessment by SEM) However, only very low encapsulation efficiencies were achieved with a maximum value of about 13% for the optimised formulation (Table 3, Ex. 1-R). Extractable porogens, which are to a certain degree water-soluble, may favorably be combined with a single emulsion-solvent evaporation process. It has been reported earlier to use poloxamers, e.g. Pluronics® F-68/F-127, as extractable porogens for a single emulsion process [Kim H., Park H., Lee J., Kim T. H., Lee E. S., Oh K. T., Lee K. C., Youn Y. S., Biomaterials, 32, 1685-1693 (2011)]. In a comparative experiment significantly higher encapsulation efficiencies were obtained with respect to the first method (I-1), but the particles thus obtained showed a very low degree of porosity (qualitative assessment by SEM, Table 3 Ex. 2-R vs. Ex. 1-R).

II. Single-Emulsion Method (o/w) to Prepare Porous Microparticles Encapsulating Small Molecule Drugs Using Different Porogenic Agents.

This example illustrates embodiments for the preparation of porous microparticles for inhalation encapsulating an API, wherein the process is a single emulsion-solvent evaporation process conducted in one-pot and comprises the use of polyvinylpyrrolidones as extractable porogenic agents.

### II-1. General Procedure for the Single-Emulsion Process

The drug (e.g. for cinaciguat 30 mg corresponding to 0.053 mmol; e.g. for budesonide 31 mg corresponding to 0.070 mmol) to be encapsulated was dissolved in 3 mL of a mixture of NMP/DCM (1:19, v/v) containing 600 mg PLGA and the porogenic agent (Pluronic® F-127 or PVP K12, K17). The resulting organic phase was then injected into 300-500 mL of an aqueous phase containing 0.1-1.0% PVA 205 or PVA 217 (w/v) as an emulsifier to form an o/w emulsion. A high-speed homogenizer (T 25 ULTRA-TURRAX®, IKA, Gießen, Germany) was used for the emulsification operated at a homogenization speed (v_{H}) of 8000-15000 rpm for 30 s. The final emulsion was subsequently stirred at 800 rpm for 5 h at room temperature to evaporate the volatiles. The particles were collected by centrifugation (4000 rpm, 10 min) and washed three times with distilled water, then lyophilized for 24 h (0.01 atm, −50° C.).

### II-2. Porous Microparticles Containing Cinaciguat

Following the general procedure of the single emulsion method as described above, porous microparticles containing cinaciguat were prepared and the specified different porogenic agents were tested under the given process conditions. The particle characteristics were determined as described in the methods section. The experiments under the given process conditions and the test results are shown in Table 4.

TABLE 4

| | | | | Volume | | | | |
|---|---|---|---|---|---|---|---|---|
| | | | | Mean | | | | |
| | | Porogen/ | | Diameter | | | | |
| | | PLGA | v_{H} | (D[4, 3]),^{#} | MMADₑ^{#} | | | Porosity/ |
| Ex. | PLGA | (w/w) | [rpm] | [μm] | (μm) | % EE^{#} | % DL^{#} | Morphology^{##} |
| 3 | 502 | 10% | 8000 | 15.74 | 5.97 | 81.7 ± 2.6 | 4.09 ± 0.13 | moderate |
| | | PVP K12 | | | | | | |
| 4 | 502H | 10% | 8000 | 18.55 | 6.20 | 79.7 ± 2.0 | 3.99 ± 0.10 | moderate |
| | | PVP K12 | | | | | | |
| 5 | 503H | 10% | 8000 | 24.17 | NA | 82.8 ± 1.7 | 4.14 ± 0.09 | moderate |
| | | PVP K12 | | | | | | |
| 6 | 502 | 20% | 8000 | 17.68 | 5.99 | 71.5 ± 2.5 | 3.58 ± 0.13 | high; large |
| | | PVP K12 | | | | | | pore size |
| 7 | 503H | 20% | 8000 | 24.71 | NA | 77.9 ± 2.8 | 3.90 ± 0.14 | high; small |
| | | PVP K12 | | | | | | pore size |
| 8 | 502 | 20% | 11000 | 13.83 | 5.80 | 68.1 ± 2.6 | 3.41 ± 0.13 | high; large |
| | | PVP K12 | | | | | | pore size |
| 9* | 503H | 20% | 15000 | 12.26 | 5.65 | 74.0 ± 3.3 | 3.70 ± 0.17 | high; small |
| | | PVP K12 | | | | | | pore size |
| 10-R | 503H | 20% | 8000 | 20.76 | NA | 88.3 ± 2.4 | 4.42 ± 0.12 | few pores on |
| | | F-127 | | | | | | the surface; |
| | | | | | | | | hollow |
| | | | | | | | | particles |
| 11-R | 502 | 50% | 8000 | 15.54 | 5.21 | 103.5 ± 4.5 | 5.18 ± 0.23 | low; |
| | | F-127 | | | | | | fragments |
| | | | | | | | | detected |
| 12-R | 502 | 50% | 9500 | 9.39 | 5.22 | 95.6 ± 4.0 | 4.78 ± 0.20 | low; |
| | | F-127 | | | | | | fragments |
| | | | | | | | | detected |
| 13-R | 502 | no | 8000 | 10.52 | n.d. | 80.9 ± 1.7 | 4.05 ± 0.13 | no pores |
| | | porogen | | | | | | |
| 14-R | 503H | no | 8000 | 15.82 | n.d. | 85.8 ± 1.6 | 4.29 ± 0.08 | no pores |
| | | porogen | | | | | | |

R: Reference example. For all experiments ~30 mg (0.053 mmol) of cinaciguat was used. For all experiments an 0.1% (w/v) aqueous PVA 205 solution (300 mL) was used (O/W ratio 3:300). Reference examples 13-R and 14-R were prepared by the described method without the use of any porogenic agent.
^{#}Given values were determined as described in the methods section.
n.d.: Values were not determined.
“NA” for “not applicable” given by the NGI software, when particle distribution is not lognormal well (regression coefficient, R <0.95), mainly due to a large D[4, 3] value.
^{##}Qualitatively assessed from SEM pictures (detailed discussion under IV-2).
*Used for in-vivo study (FIG. 9 and 10).

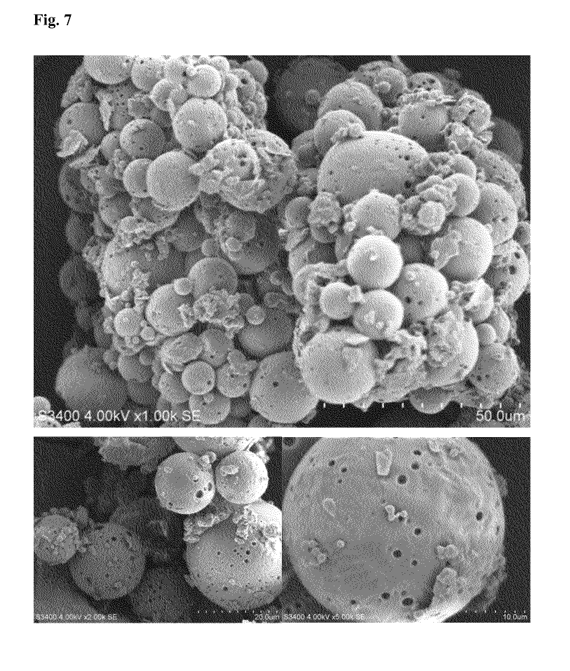

Table 4 shows that good drug encapsulation efficiencies and favorable particle properties, such as low MMAD values were achieved by the described method. By adjusting the homogenization speed (v_{H}) during the emulsification period (e.g. Ex. 6 and 7 vs. Ex. 8 and 9) as well as the viscosity of the polymer matrix former (PLGA 502H<PLGA 503H; e.g. Ex. 4 vs. 5) microparticle size may be controlled, while the aerodynamic diameters of the microparticles were in a similar range. Porosity of the microparticles increases with increasing amount of porogen used. Microparticles which were prepared by using an alternative extractable porogen (poloxamer F-127, reference Ex. 10-R; 11-R, 12-R) have considerable lower porosity and also show significant aggregation/fragmentation (FIG. 7).

### II-3. Porous Microparticles Encapsulating Budesonide

Following the general procedure of the single emulsion method as described above, porous microparticles containing budesonide were prepared and different porogenic agents were tested under the given process conditions. The particle parameters were determined as described in the methods section. The experiments under the given process conditions and the test results are shown in Table 5.

TABLE 5

| | | | | Volume | | | | |
|---|---|---|---|---|---|---|---|---|
| | | | | mean | | | | |
| | | Porogen/ | PVA (w/v) | diameter | | | | |
| | | PLGA | (O/W ratio | D[4, 3]^{#}, | MMADₜ^{#} | | | |
| Ex. | PLGA | (w/w) | v/v) | [μm] | [μm] | % EE | % DL^{#} | Porosity |
| 15 | 503H | 20% PVP | 0.1% | 22.80 | 10.19 | 50.3 ± 0.6 | 2.52 ± 0.03 | high; |
| | | K12 | PVA205 | | | | | large pore |
| | | | (3:300) | | | | | size |
| 16 | 503H | 15% PVP | 0.1% | 16.52 | 7.76 | 54.2 ± 0.3 | 2.71 ± 0.02 | moderate |
| | | K12 | PVA205 | | | | | |
| | | | (3:300) | | | | | |
| 17 | 503H | 10% PVP | 0.1% | 14.82 | 10.94 | 45.4 ± 0.5 | 2.27 ± 0.03 | no pores |
| | | K12 | PVA205 | | | | | on the |
| | | | (3:300) | | | | | surface |
| 18 | 503H | 15% PVP | 0.1% | 18.05 | 9.99 | 43.1 ± 2.7 | 2.16 ± 0.14 | moderate |
| | | K12 | PVA205 | | | | | |
| | | | (3:400) | | | | | |
| 19 | 503H | 15% PVP | 0.1% | 17.77 | 11.37 | 55.0 ± 0.4 | 2.75 ± 0.02 | low; |
| | | K12 | PVA205 | | | | | small |
| | | | (3:500) | | | | | pore size |
| 20 | 503H | 15% PVP | 0.5% | 16.46 | 10.35 | 60.2 ± 0.5 | 3.01 ± 0.03 | low; |
| | | K12 | PVA205 | | | | | small |
| | | | (3:500) | | | | | pore size |
| 21 | 503H | 15% PVP | 1.0% | 19.98 | 10.87 | 50.8 ± 1.5 | 2.54 ± 0.08 | low; |
| | | K12 | PVA205 | | | | | small |
| | | | (3:500) | | | | | pore size |
| 22 | 503H | 15% PVP | 0.5% | 16.14 | 8.68 | 51.6 ± 2.6 | 2.58 ± 0.13 | low; |
| | | K12 | PVA205 | | | | | small |
| | | | (3:300) | | | | | pore size |
| 23 | 503 | 15% PVP | 0.5% | 13.48 | 6.30 | 58.5 ± 1.2 | 2.93 ± 0.06 | high; |
| | | K12 | PVA205 | | | | | large pore |
| | | | (3:300) | | | | | size |
| 24 | 503H | 15% PVP | 0.5% | 6.44 | n.d. | n.d. | n.d. | no pores |
| | | K12 | PVA217 | | | | | on the |
| | | | (3:500) | | | | | surface |
| 25 | 503H | 20% PVP | 0.5% | 13.48 | 8.80 | 61.9 ± 1.4 | 3.10 ± 0.07 | no pores |
| | | K12 | PVA217 | | | | | on the |
| | | | (3:500) | | | | | surface |
| 26 | 503H | 25% PVP | 0.5% | 14.78 | 9.36 | 54.5 ± 0.3 | 2.73 ± 0.02 | few; very |
| | | K12 | PVA217 | | | | | small |
| | | | (3:500) | | | | | pore size |
| 27 | 503H | 20% PVP | 0.5% | 14.80 | 8.73 | 50.8 ± 1.5 | 2.54 ± 0.08 | n.d. |
| | | K17 | PVA217 | | | | | |
| | | | (3:500) | | | | | |
| 28 | 503H | 20% PVP | 0.5% | 12.83 | 9.15 | 44.0 ± 0.8 | 2.20 ± 0.04 | n.d. |
| | | K30 | PVA217 | | | | | |
| | | | (3:500) | | | | | |
| 29-R | 503 | 10% PEG | 0.1% | 19.11 ± 0.11 | 11.05 ± 0.25 | 41.2 ± 0.4 | 2.06 ± 0.02 | no pores |
| | | 4000 | PVA205 | | | | | on the |
| | | | (3:300) | | | | | surface |
| 30-R | 503 | 15% PEG | 0.1% | 13.19 ± 0.06 | 5.82 ± 0.11 | 43.7 ± 0.3 | 2.19 ± 0.02 | hollow; |
| | | 4000 | PVA205 | | | | | large |
| | | | (3:300) | | | | | pores |

R: Reference example.
n.d.: values were not determined.
For all experiments 31.0 mg (0.070 mmol) of budesonide was used. For all experiments the homogenization speed was v_{H} = 15000 rpm.
^{#}Given values were determined as described in the methods section.

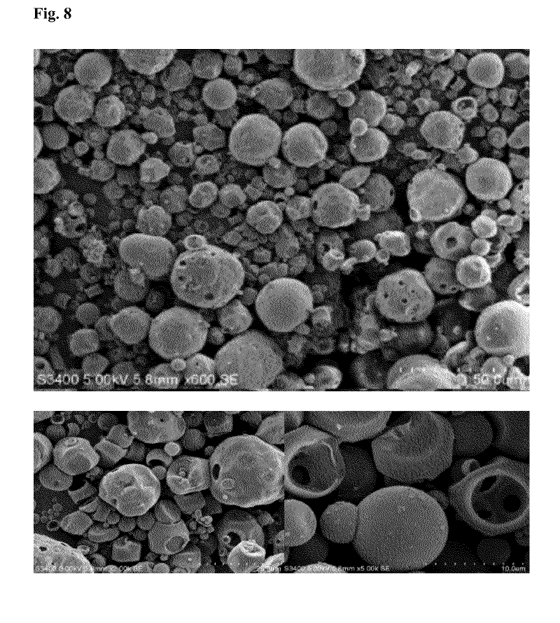

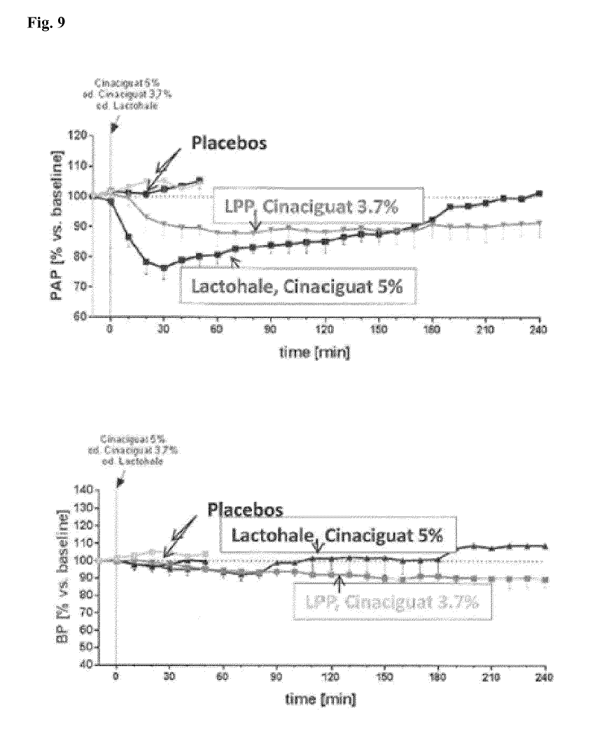

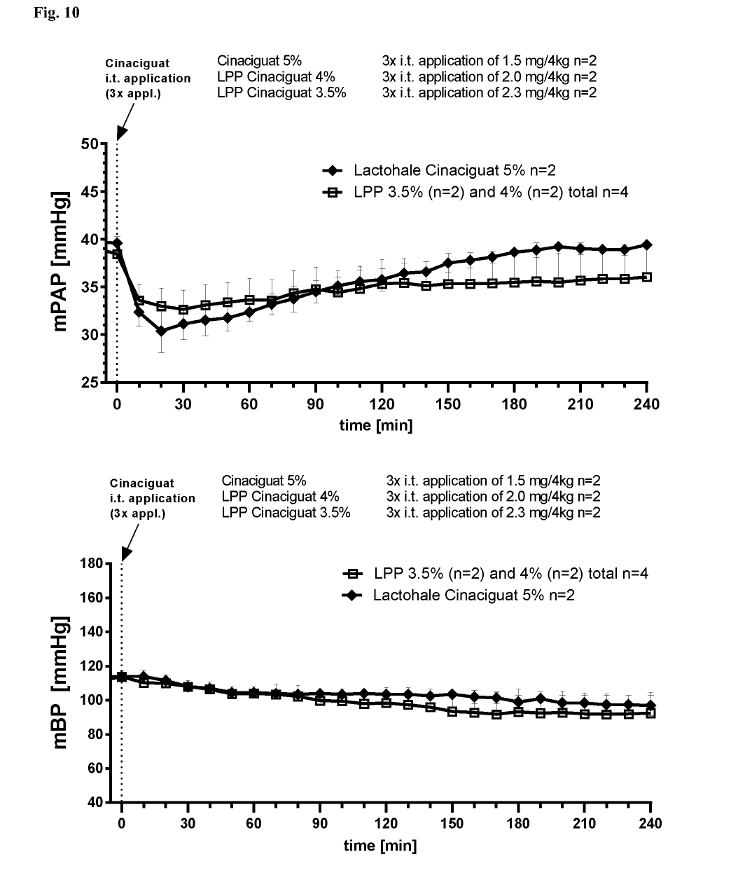

Table 5 shows that in general lower encapsulation efficiencies were observed for budesonide containing microparticles when compared to cinaciguat containing microparticles (cf. Table 4). Porosity of the microparticles increases with increasing amount of porogen used (e.g. Ex. 3 vs. Ex. 2 vs. Ex. 1). By adjusting the ratio between aqueous phase/organic phase a fine-tuning of the aerodynamic parameters and porosity is possible (e.g. Ex. 16, 18 and 19), also by chosing the type of emulsifier (PVA 205 vs. PVA 217; e.g. Ex. 21 vs. Ex. 24-26) while the concentration of the emulsifer PVA shows only a minor influence (Ex. 19, 20, 21). A lower hydrophilicity of the polymer matrix former (PLGA 503<503H) also influences the size, aerodynamic properties and porosity of the microparticles (e.g. Ex. 22 vs. 23). Microparticles which were prepared by using an alternative extractable porogen (PEG 4000, reference Ex. 29-R, 30-R) show poor control of the microparticle porosity and also have a very unfavorable irregular shape and morphology (FIG. 8).

### III. Evaluation of the Drug Release Profile and the Aerodynamic Properties of the Porous Microparticles

This example illustrates embodiments of the porous microparticles encapsulating an API, which are obtained via a process according to the invention, and thereof derived pharmaceutical compositions for pulmonary drug delivery. Specific embodiments have a sustained release profile, where the API is released over a specified time period.

### III-1. In Vitro Evaluation of the Drug Release Profiles of the Porous Microparticles

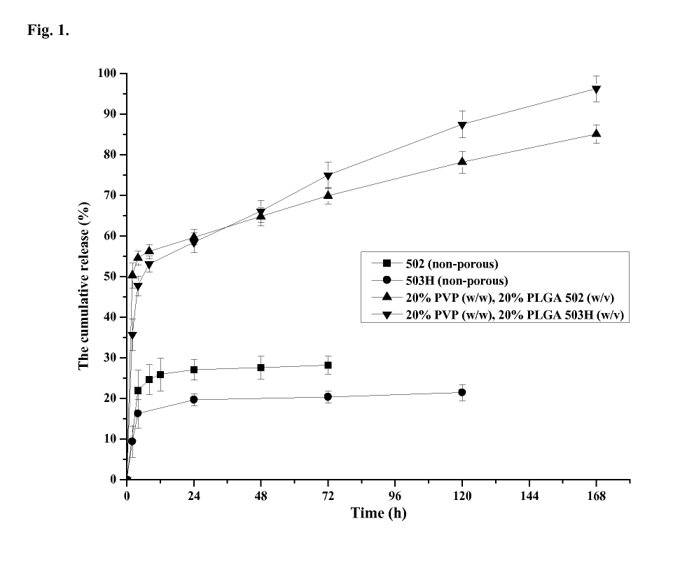

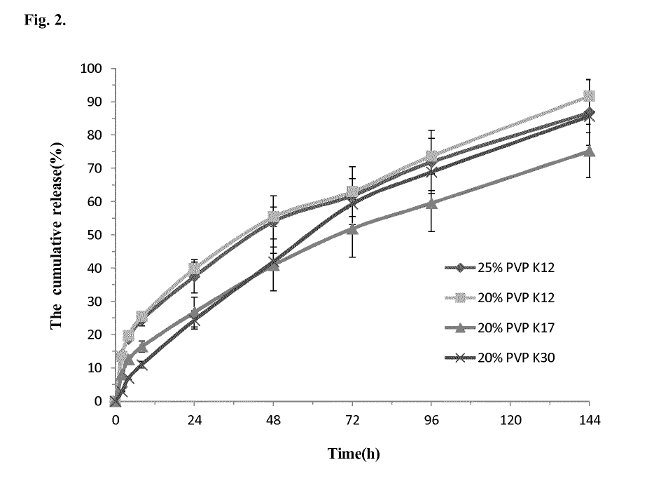

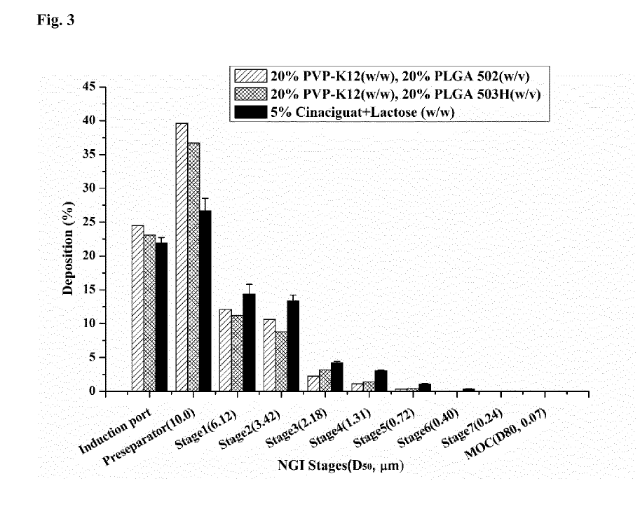

The resulting microparticles prepared under the conditions described above (Ex. II-1 to II-3), were tested with regard to their in vitro release behavior under non-sink conditions as described in the methods section (method E)). The release rate of the encapsulated API may be evaluated in vitro to identify those formulations having a desired release rate in a given amount of time. Thus, the level of porosity for the respective polymer type can be used to adjust the amount of pharmaceutical agent released after a certain period of time, and particles having a desired release profile can be further analyzed in vivo. FIGS. 1 and 2 are graphs of percent of API released in vitro at the indicated time points from the optimised formulations.

FIG. 1 shows the in vitro release rate of the optimised microparticle formulations comprising cinaciguat as API, which were prepared as described in example II-2, experiments 8 and 9. The in vitro release rate can be used to evaluate the desired level of sustained release in vivo. Microparticles which do not have a sufficient degree of porosity show a non-favorable release profile (reference Ex. 13-R and 14-R without the use of a porogenic agent).

FIG. 2 shows the in vitro release rate of the optimised microparticle formulations comprising budesonide as API, which were prepared as described in example II-3, experiments 25, 26, 27 and 28.

### III-2. In Vitro Evaluation of the Aerodynamic Properties of the Porous Microparticles

The resulting microparticles prepared under the conditions described above (II-1 to II-3), were tested with regard to their in vitro aerosolization performance from a dry powder inhaler (Cyclohaler, (PH&T, Milano, Italy) using VCAPS Plus HPMC capsules (Capsugel, Greenwood, USA) as described in the methods (method F). The content of drug loaded LPP powder on each stage was detected by HPLC and the aerosol performance parameters, FPF %, MMADₑ and GSD values, were calculated by the NGI software and tabulated in Table 6 (The MMADₑ values were already given in Table 4).

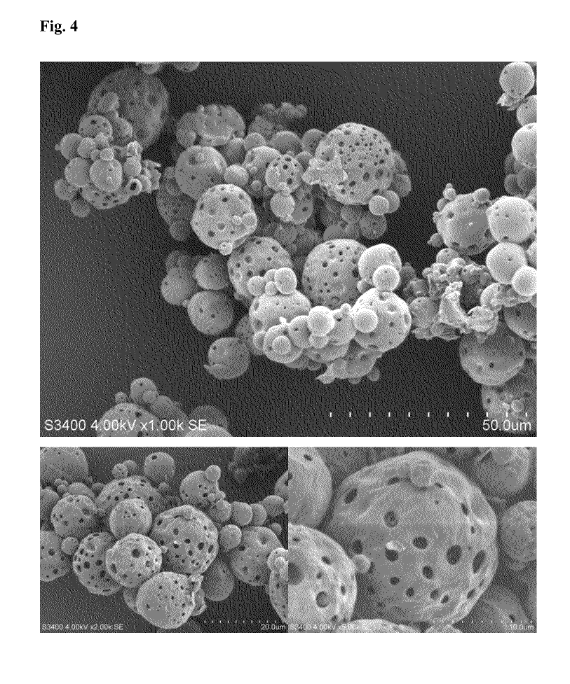

FIG. 3 is a depiction of the in vitro aerodynamic diameter distribution and the deposition of the optimised particle formulations comprising cinaciguat as API, which were prepared as described in example II-2, experiments 8 and 9, in the NGI model compared with a conventional lactose-based powder blend.

TABLE 6

| | | PLGA/ | MMADₑ^{#} | FPF^{#} | |
|---|---|---|---|---|---|
| Ex. | API | porogen | [μm] | [%] | GSD^{#} |
| 8 | cinaciguat | PLGA 502/ | 5.80 | 26.2 | 1.63 |
| | | 20% PVP K12 | | | |
| 9* | cinaciguat | PLGA 503H/ | 5.65 | 24.7 | 1.86 |
| | | 20% PVP K12 | | | |
| 22 | budesonide | PLGA 503H/ | NA | 21.0 | NA |
| | | 15% PVP K12 | | | |
| 23 | budesonide | PLGA 503/ | NA | 23.4 | NA |
| | | 15% PVP K12 | | | |
| Lactohale-R | cinaciguat | none | 4.82 | 36.0 | 2.04 |

*Used for in-vivo study (FIG. 9 and 10).

The above results show that porous microparticles according to the invention exhibit fine particle fractions (˜25% for Cinaciguat, ˜22% for Budesonide) that are in the range of commonly used and marketed dry podwer inhaler formulations (which is in the range of ˜20%-30%, Muralidharan et al., Expert Opinion Drug Deliv., November 2014) in which the API is applied to the patient in pure form or blended with solid excipients such as e.g. lactose or mannitol. Using the Cyclohaler device, a direct comparison of FPF obtained with Cinaciguat porous microparticles according to the invention vs. FPF obtained from a Cinaciguat-Lactohale® (Lactohale LH 200®/Lactohale LH 300®, 80/20; DFE Pharma, Goch, Germany) powder blend, reveals a difference (FPF microparticles ˜25%, FPF Lactohale powder blend ˜36%), however, both types of formulations are comparable in a way that the FPF of both formulations is in an acceptable range for pulmonary application.

FIG. 3 shows the percentages of particles mass in each of the cut-off plates of the NGI. A typical pattern for solid API formulations when emitted from a dry powder inhaler can be observed with the majority of particles ending up in the preseparator (˜35-40% of particle mass), followed by the mass of particles in the induction port (˜20-25% of particle mass). The fine particle fraction (sum of stages 1-5), as also shown in Table 4, is about 25%.

### Reference Example: Lactohale-R

The Lactohale® reference formulation was prepared by blending Lactohale LH 200® (8 parts) with Lactohale LH 300® (2 parts) in a Turbula powder blend mixer (Bachofen AG Maschinenfabrik, Muttenz, Switzerland). Then, Cinaciguat was added and blended to a final concentration of 5% (m/m). In order to show that the blending process resulted in a homogenous Cinaciguat powder blend, content uniformity (CUT) according to USP was determined. Respective CUT results met USP requirements.

### IV. Evaluation of the Morphology of the Porous Microparticles

This example illustrates embodiments of the porous microparticles containing an API, which are obtained via a process according to the invention, and which are suitable to be administered in the form of pharmaceutical compositions for pulmonary drug delivery.

### Iv-1. Evaluation of the Physical Form of the API

Thermodynamic calorimetry (DSC) as well as X-ray powder diffraction (X-RPD) can be used to evaluate the physical form and the encapsulation state of the pharmaceutically active agent.

### IV-2. Evaluation of the Surface Morphology of the Porous Microparticles

Scanning electron microscope (SEM) photomicrographs can be used to reveal the surface characteristics, especially desired porosity, as well as uniformity or agglomeration of the porous microparticles.

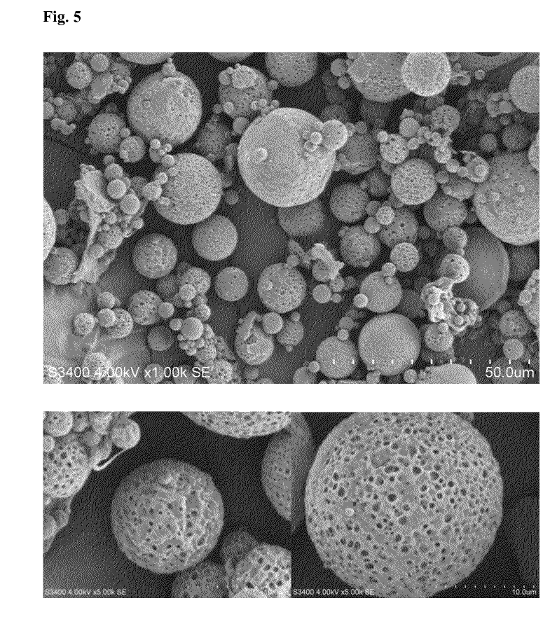

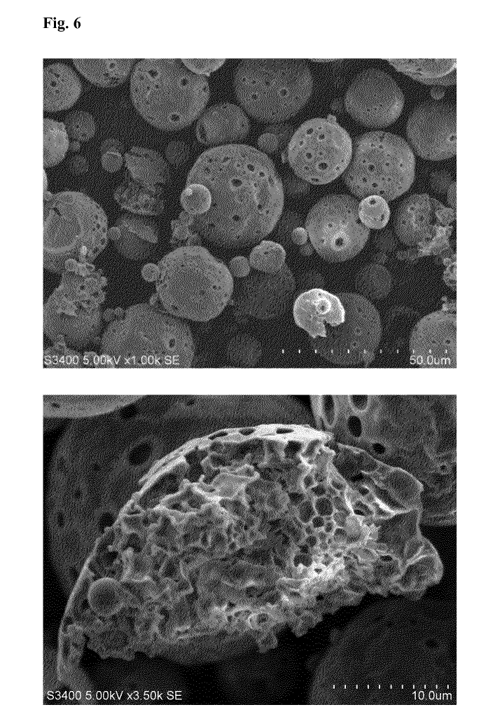

FIGS. 4 and 5 are SEM photographs (recorded as specified in the methods section) of the porous microparticles comprising cinaciguat as API, which were obtained by the single emulsion method according to the invention as described (example II-2, experiments 8 and 9). The microparticles are spherical in shape and show favorable uniformity as well as an even distribution of the pores on the particle surface.

FIG. 6 is an SEM photograph of porous microparticles comprising cinaciguat as API, which were obtained by the single emulsion method according to the invention (formulation not shown in Table 4; 50% PVP K12/PLGA 503 was used). Some of the microparticles from this experiment are cracked, revealing the high degree of inner porosity of the particles. Such an inner porous structure is beneficial in order to achieve sufficiently low density, thus sufficiently low MMADₑ (<10 μm, ideally <5 μm) to allow for lung deposition after inhalation of the microparticles, with at the same time sufficiently high Dᵥ (>10 μm, ideally >15 μm) in order to escape lung macrophage uptake and to slow down mucociliary clearance.

FIG. 7 is an SEM photograph of microparticles, which were obtained by a single emulsion based process according to the general procedure of II-1, but using poloxamer pluronic F-127 as an extractable porogenic agent (II-2, Table 4, reference experiment 12-R). The microparticles are spherical in shape with few smaller pores on the particle surface. A high degree of aggregation and fragmentation is detected and several smaller fragments (e.g. API crystals) of different morphology and shape adhere to the particle surfaces and agglutinate the single microparticles. Microparticles, which do not show a regular and uniform particle morphology and distribution of the particles, and/or where the formulations are not reliably reproducible, cannot guarantee a reliable biopharmaceutical performance of the API (API dissolution, followed by API permeation into lung tissue and/or into the blood vessel and/or into the blood stream) and thus cannot guarantee a reliable therapeutic effect of the API in patients. Therefore, formulations which cannot be reproducibly manufactured are not suitable to be used as an inhalable pharmaceutical composition for disease therapy.

FIG. 8 is an SEM photograph of microparticles, which were obtained by a single emulsion based process according to the general procedure of II-1, but using PEG4000 as extractable porogenic agent (II-3, Table 5, reference experiment 29-R). The particles from this method have a very unfavorable irregular shape and morphology and are not suitable for the purpose of an inhalable pharmaceutical composition.

### V. In Vivo Drug Release Profile and Therapeutic Efficacy of the Porous Microparticles

This example illustrates embodiments of the porous microparticles encapsulating an API, which are obtained via a process according to the invention, and thereof derived pharmaceutical compositions for pulmonary drug delivery. A specific embodiment shows a sustained therapeutic efficacy for pulmonary drug delivery in an in vivo inhalation model. A specific embodiment shows a sustained antihypertensive efficacy in a pulmonary arterial hypertension animal model.

### V-1. In Vivo Pharmacodynamic Study of Pulmonary Arterial Hypertension

Porous microparticles, which were identified from the in vitro release experiments to have the desired release profile, can be further assessed in a selected in vivo animal model of pulmonary drug delivery.

The resulting porous microparticles prepared under the conditions described above (II-1 to II-2) and which had been evaluated in respect of their release profile, were tested in an in vivo pharmacodynamic study in an animal model (minipig) of pulmonary arterial hypertension, as described in the methods section. The optimised microparticle formulation (example II-2, Table 4, Ex. 9; 3.7% Cinaciguat; applied dose 2.0 mg/4 kg piglet) comprising cinaciguat as an API was compared with a standard cinaciguat-lactose based powder blend (Cinaciguat 5%; applied dose 1.5 mg/4 kg piglet). The selected formulations were loaded in a dry powder insufflator device and administered to the minipigs via intratracheal application (n=2). Over the duration of the experiment (240 min) pulmonary arterial blood pressure (PAP) as well as systemic arterial blood pressure (BP) were continuously monitored as shown in FIGS. 9 and 10. Before the application of the Cinaciguat formulations pure matrix/formulation applications were performed to differentiate the pure application effects from the drug effects. Data are shown as mean+/−SEM of 2 independent experiments per formulation (Cinaciguat 3.7%: FE495KK, FE497JK; Cinaciguat 5%: FE493JK; FE496JK).

FIGS. 9 and 10 show a depiction of the recorded pulmonary arterial blood pressure (PAP) and the systemic arterial blood pressure (BP) of an in vivo minipig model following administration of the optimised microparticle formulation comprising cinaciguat as API (prepared as described in example II-2, Table 4, Ex. 9) at two doses (as described under Methods, J, a) and b), as well as of the control formulation of a standard cinaciguat-lactose based powder blend (table 6, Lactohale-R). With both formulation types Cinaciguat reduced PAP and systemic blood pressure, however, to a different extent and duration. With the LPP micropaticle formulation, the decrease in the PAP was diminished in its maximal effect, when compared to the Lactohale powder blend. At the same time lowering of PAP caused by the LPP formulation was sustained over the entire 4 hours duration of the experiment, whereas PAP reduction observed with the Lactohale powder blend already began to decrease ˜30 minutes post administration (FIGS. 9 and 10) Also with respect to systemic BP a slight reduction was observed for both formulations. Again with the LPP formulation, the duration of blood pressure reduction lasted over the entire 4 hours duration of the experiment, whereas with the Lactohale formulation, systemic blood pressure went back to baseline after about 2 hours post administration (FIG. 9). At the higher dose this effect on BP was less pronounced (FIG. 10). Altogether, the data presented in FIGS. 9 and 10 demonstrate a sustained in vivo release of Cinaciguat from the optimized LPP microparticle formulation.

### Histopathology

In the trachea, epithelial atrophy, degeneration and erosion, focally with intraluminal detritus and cells was detected, most likely due to the experimental procedure. At the tracheal bifurcation, the epithelium appeared normal. Furthermore, alveolar macrophages were seen in the lungs, occasionally with vacuolated cytoplasm. Additional pulmonary findings, sometimes seen also in other organs (e.g. liver or kidney) were focal inflammatory infiltrates.

All findings detected in the organs/tissues evaluated during histopathology, are not assessed to be adverse lesions related to the exposure of the test particles. The findings are known from controls of other studies and therefore assessed to be of spontaneous nature. If the macrophage vacuoles are related to the test substance, they are an indication of normal cellular function.

Findings seen during histopathology were for example focal inflammatory infiltrates in various organs/tissues or focal pigment deposition. All findings detected in the organs/tissues evaluated, were assessed to be of spontaneous nature and not related to the exposure to the particles.

1. A process for the preparation of porous microparticles for pulmonary drug delivery comprising a matrix material and a pharmaceutically active agent, the process comprising the steps
(i) preparing an o/w emulsion, wherein
a first phase (a) comprising a pharmaceutically active agent, a matrix material, a porogenic agent and a volatile solvent, is emulsified with
a second, aqueous phase (b), optionally comprising an emulsifying agent,
(ii) optionally stirring the o/w emulsion resulting from step (i),
(iii) removing the volatile solvent,
(iv) separating the porous microparticles from the remaining phase resulting from step (iii),
(v) optionally drying the porous microparticles resulting from step (iv),
wherein the porogenic agent in step (i) is polyvinylpyrrolidone and/or a polyvinylpyrrolidone derivative.

2. A process according to claim 1, wherein the pharmaceutically active agent has a solubility in water of less than 1 mg/mL, preferably less than 0.1 mg/mL.

3. A process according to claim 1, wherein the matrix material is a biocompatible and/or biodegradable polymer, selected from the group consisting of poly(lactide-co-glycolide), poly(lactide), or poly(glycolide) and derivatives thereof.

4. A process according to claim 3, wherein the matrix material comprises mainly free terminal carboxy groups.

5. A process according to claim 3, wherein the matrix material is poly(lactide-co-glycolide) acid with a mole ratio of 48 to 52 mole % lactide and 48 to 52 mole % glycolide, wherein the amount of lactide and glycolide is 100 mole %.

6. A process according to claim 1, wherein the solvent is selected from the list of the following solvents: dichloromethane, cyclohexane, hexane, methylbutylketone, N-methylpyrrolidone, tert.-butylmethylether, ethyl acetate, diethylether, heptane, pentane or a mixture thereof.

7. A process according to claim 1, wherein the porogenic agent is used in a ratio of from 5% to 50%, preferably of from 10% to 30%, more preferably from 15 to 25%, by weight (w/w), relative to the matrix material.

8. A process according to claim 1, wherein polyvinylpyrrolidone with a K value of from 12 to 40, preferably of from 12 to 17, is used as a porogenic agent.

9. A process according to claim 1, wherein the emulsification in step (i) is performed at a homogenization speed (v_{H}) of from 6000 to 15000 rpm.

10. Porous microparticles for pulmonary drug delivery comprising a matrix material and a pharmaceutically active agent, obtainable via a process according to claim 1.

11. Porous microparticles according to claim 10, in which the pores pervade the entire volume of the microparticle.

12. Porous microparticles according to claim 10 with an MMAD value between 1 μm to 10 μm, and a geometric particle size of greater than 10 μm.

13. Porous microparticles according to claim 10, wherein the pharmaceutically active agent is selected from the group comprising cGMP elevating agents e.g. sGC stimulators and activators, PDE inhibitors, IP receptor agonists, endothelin receptor antagonists, HNE inhibitors, signal transduction cascade inhibitors, antithrombotic agents and vasodilators.

14. Porous microparticles according to claim 10, with a sustained release of the pharmaceutically active agent of less than 60% after 12 hours.

15. Pharmaceutical composition comprising porous microparticles as defined in claim 10 and optionally one or more pharmaceutically acceptable excipients.

16. The pharmaceutical composition of claim 15 further comprising one or more additional therapeutic agents selected from the group consisting of cGMP elevating agents e.g. sGC stimulators and activators, PDE inhibitors, IP receptor agonists, endothelin receptor antagonists, HNE inhibitors, signal transduction cascade inhibitors, antithrombotic agents and vasodilators.

17. The pharmaceutical composition of claim 15 for use in the treatment and/or prevention of diseases.

18. The pharmaceutical composition according to claim 15 for use in the treatment and/or prevention of pulmonary hypertension, chronic obstructive pulmonary disease (COPD), pulmonary fibrosis and lung cancer.

19. The pharmaceutical composition according to claim 15 for the treatment of pulmonary arterial hypertension comprising porous microparticles comprising an effective amount of an sGC activator.

20. Use of polyvinylpyrrolidone and/or a polyvinylpyrrolidone derivative as an extractable porogenic agent for the preparation of porous microparticles for pulmonary drug delivery via an emulsion solvent evaporation process.

21. Use according to claim 20, wherein the emulsion solvent evaporation process is a single emulsion-solvent evaporation process.
